(19) United States
(12) Patent Application Publication
(10) Pub. No.: US 2011/0245094 A1
(43) Pub. Date: Oct. 6, 2011

(54) DETECTION OF MICROBIAL NUCLEIC ACIDS

(75) Inventors: Brian K. Washburn, Boston, MA (US); Andrew E. Levin, Boston, MA (US)

(73) Assignee: IMMUNETICS, INC., Boston, MA (US)

(21) Appl. No.: 12/990,761

(22) PCT Filed: May 1, 2009
(86) PCT No.: PCT/US09/42578

§ 371 Date: Jun. 10, 2011

Related U.S. Application Data
(60) Provisional application No. 61/050,188, filed on May 2, 2008.

(51) Int. Cl. C40B 30/04; C12N 1/15; C07H 21/04; C40B 40/06

(52) U.S. Cl. 506/9; 435/254.11; 536/23.1; 536/24.33; 506/16

(57) ABSTRACT

The present invention features, inter alia, compositions and methods useful for identifying one or more types of microorganisms, if and when present, in a sample or plurality of samples (e.g., in one or more samples tested in parallel). More specifically, the present compositions and methods can be used in, e.g., determining whether a subject has a microbial infection (e.g., a bacterial, fungal, protozoal, or viral infection), determining the identity of the microbe(s) causing the infection, and/or determining, or helping to determine, an appropriate anti-microbial treatment regimen for a subject identified as having an infection (e.g., an appropriate antibiotic, anti-fungal, anti-viral, or other treatment regimen).

### CROSS-REFERENCE TO RELATED APPLICATIONS

This application claims the benefit of the priority date of U.S. Application No. 61/050,188, which was filed May 2, 2008. For the purpose of any U.S. application that may issue based on the present international application, the content of this prior provisional application is hereby incorporated by reference in its entirety.

### TECHNICAL FIELD

This invention relates to compositions and methods useful in the detection, identification, and treatment of microbial infections.

### BACKGROUND

Two of the major causes of death from healthcare-associated infections are bloodstream infections and pneumonia (Klevens et al., Public Health Rep. 122:160-166, 2007). Bloodstream infections can cause severe sepsis, which has a very high mortality.

In addition to bloodstream infections, hospital-acquired pneumonia (HAP) is one of the most common causes of death in the intensive care unit (Klevens et al., Public Health Rep. 122:160-166, 2007). Ventilator-associated pneumonia (VAP), is the most common and well studied type of HAP (Patel et al., Semin Respir Crit Care Med. 23:415-25, 2007). HAP is one of the primary reasons antibiotics are prescribed in the ICU (Brun-Buisson, Semin Respir Crit Care Med. 23:457-69, 2002; Chastre et al., Clin Infect Dis. 43 Suppl 2:S75-81, 2006). A wide variety of bacteria are associated with hospital-acquired pneumonia, including S. pneumoniae, H. influenzae, S. aureus (often MRSA (methicillin resistant S. aureus)), Pseudomonas, Acinetobacter, Enterobacteriaceae (e.g., Klebsiella, Enterobacter, typically β-lactamase producing), and S. maltophilia (Brun-Buisson, Semin Respir Crit Care Med. 23:457-69, 2002; Weber et al., Infect Control Hosp Epidemiol, 28:825-31, 2007; Kollef, Eur. J. Clin. Microbiol. Infect. Dis. 24:794-803, 2005; Hunter, Postgrad Med. J. 82:172-8, 2006; Lambotte et al., Chest 122:1389-99; 2002; Martin, Medscape Pulmonary Medicine 9 2005; Fagon et al., Am. Rev. Respir. Dis. 139:877-84, 1989; Park, Respir. Care 50:742-63, 2005). The bacteria causing hospital-acquired pneumonia are frequently highly antibiotic resistant (Brun-Buisson, Semin Respir Crit Care Med. 23:457-69, 2002; Kollef, Eur. J. Clin. Microbial. Infect. Dis. 24:794-803, 2005), and the use of incorrect antibiotics is associated with poor outcome for HAP (Brun-Buisson, Semin Respir Crit Care Med. 23:457-69, 2002; Martin, Medscape Pulmonary Medicine 9, 2005; Bodmann, Chemotherapy 51:227-33, 2005; Kollef, Intensive Care Medicine 29:147-149, 2003; Chastre, Surg. Infect. (Larchmt) 7 Suppl 2:S81-5, 2002; Bowton, Chest 122:401-2, 2002). Bacteria such as S. pneuminiae and Legionella spp. may also be causes of community-acquired pneumonia.

For HAP, the microbial investigation is complex and there is no “gold standard” (Bowton, Chest 122:401-2, 2002). Blood samples are often negative and frequently do not identify the same organisms found in respiratory samples (Chastre et al., Am. J. Respir. Crit. Care Med. 165:867-903, 2002; Luna et al., Chest 111:676-85, 1997). Respiratory sample types for culture analysis of HAP include sputum, endotracheal aspirates, bronchoalveolar lavage (BAL) or protected specimen brush (PSB). Detection of bacteria in lower respiratory samples does not provide definitive evidence of pneumonia (Patel, et al., Semin. Respir. Crit. Care Med. 23:415-25, 2002; Brun-Buisson, Semin Respir Crit Care Med. 23:457-69, 2002; Chastre et al., Am J Respir Crit Care Med. 165:867-903, 2002; Fagon, Semin Respir Crit Care Med. 27:34-44, 2006), so quantitative cultures are often used, with cutoffs depending on the type of sample (e.g., 10³ cfu/ml for PSB, ˜10⁴ cfu/ml for BAL, and 10⁵-10⁶ cfu/ml for endotracheal aspiration) (Chastre et al., Am J Respir Crit. Care Med. 165:867-903, 2002; San Pedro, Chest 119:385 S-390S, 2001). Growth below these cutoffs is assumed to reflect colonization or contamination. The American Thoracic Society and the Infectious Diseases Society of America recommend quantitative or semiquantitative methods (Am. J. Respir. Crit. Care Med. 171:388-416, 2005).

### SUMMARY

The present invention features, inter alia, compositions and methods useful for identifying one or more types of microorganisms, if and when present, in a sample or plurality of samples (e.g., in one or more samples tested in parallel). More specifically, the present compositions and methods can be used in, for example, determining whether a subject has a microbial infection (e.g., a bacterial, fungal, protozoal, or viral infection), determining the identity of the microbe(s) causing the infection, and/or determining, or helping to determine, an appropriate anti-microbial treatment regimen for a subject identified as having an infection (e.g., an appropriate antibiotic, anti-fungal, anti-viral, or other treatment regimen). Thus, as used herein, a “microorganism” can be a bacterium, fungus, protozoa or virus. Accordingly, the present compositions and methods can be used in diagnosing and treating subjects (e.g., humans) with a variety of infections including, for example, “flus” and bacteremias such as respiratory infections, cutaneous infections, sepsis, and septic shock. For example, the methods encompass diagnosing and treating subjects for hospital-acquired pneumonia (HAP (e.g., ventilator-associated pneumonia (VAP)). Any of the present methods can include a step of identifying a subject in need of diagnosis and/or treatment.

In one aspect, the invention features methods for identifying a microorganism in at least two samples, in parallel. The methods can include the steps of: providing a first nucleic acid sample from a first source; providing a second nucleic acid sample from a second source; amplifying, if present, at least one selected region of nucleic acid sequence in each of the first and second nucleic acid samples, thereby generating amplified first and second nucleic acids; providing an array of detection oligonucleotides, wherein at least one oligonucleotide hybridizes to an amplified first or second nucleic acid when a sequence that is sufficiently complementary to the oligonucleotide is present in the amplified first or second nucleic acid; contacting the array with the amplified first and second nucleic acids; and performing an assay to detect hybridization between one or more of the detection oligonucleotides on the array and one or more of the amplified first and second nucleic acids. As hybridization occurs, it may be said to generate a hybridization pattern with respect to the detection oligonucleotides and one can thereby identify a microorganism in the first source or the second source. The hybridization pattern is generated by virtue of the binding that occurs between the various amplified nucleic acids and the various detection oligonucleotides of the array. In any of the embodiments described herein, and as discussed further below, the oligonucleotides can be arrayed on a solid support that is porous and therefore allows “flow through” of a sample applied thereto. Flow through may be assisted by vacuum, which is advantageous because it reduces the time required to analyze the samples.

The method can be configured to identify one or more (e.g., one, two, three, four, five, six, seven, eight, nine, 10, 11, 12, 13, 14, 15, 16, 17, 18, 19, or 20 or more) different microorganisms in each of one or more (e.g., one, two, three, four, five, six, seven, eight, nine, 10, 11, 12, 13, 14, 15, 20, 25, or 30 or more) different samples. The method can be configured to identify the one or more different microorganisms in a single sample or the method can be configured to identify a single type of microorganism in each of two or more (e.g., two, three, four, five, six, seven, eight, nine, 10, 11, 12, 13, 14, 15, 20, 25, or 30 or more) different sources.

Samples can be tested singly, but an advantage of the present methods is the ability to assess multiple samples essentially simultaneously or in parallel. Further, the selection of detection oligonucleotides can be varied to allow one to assess the nucleic acid content in several different samples obtained from the same subject (e.g., samples obtained at different times (e.g., over the course of treatment) or from different locations (e.g., a blood sample and a sample obtained by bronchial/alveolar lavage)). For example, nucleic acid can be extracted from a single sample of blood from a single subject at a single time and contacted to a plurality of detection oligonucleotides to simultaneously identify one or more microorganisms (one or more microbial nucleic acids), the genotype of one or more particular microorganisms, and/or the presence or absence of one or more antibiotic resistance or virulence genes within one or more of the microorganisms. For example, the method can be configured such that the identity of Klebsiella pneumonia can be determined as well as whether or not the bacterium contains a gene encoding a KPC-1 or KPC-2 carbapenemase. As noted, multiple samples from the same patient or different patients may be tested at the same time.

In an exemplary implementation, the array is configured to include two or more distinct sets of detection oligonucleotides (e.g., each column or row contains a different set of detection oligonucleotides, or one or more quadrants of a array contain a different set of detection oligonucleotides). In some embodiments, an array can be configured such that it includes two or more identical sets of detection oligonucleotides. The detection oligonucleotide sets can, e.g., contain one or more detection oligonucleotides useful for identifying different microbes (e.g., different fungi, different bacteria, different protozoa, or different viruses), different species of a given type of microorganism, different strains of a specific species of microorganism, an antibiotic resistance or virulence gene present within a microorganism, and/or any combination of the foregoing. The sets can be arrayed in parallel rows or columns, and generally will be spaced from one another such that crossover or mixing of different samples applied to the array, in parallel, is minimized or eliminated. The amplified nucleic acids from at least two different sources can be contacted to each distinct or identical oligonucleotide set in parallel, thereby allowing simultaneous determination of multiple parameters.

In some embodiments, an array can be configured for use in a “checkerboard”-type assay. The checkerboard-type assay can be used to identify multiple parameters in a single source. For example, an array can be configured to contain two or more columns of detection oligonucleotides, each column containing two or more of the same oligonucleotide. A device containing channels can be placed on top of the array such that one oligonucleotide from each column is contained within a single channel of the device. As such, two or more amplified nucleic acid samples can be contacted to the array in parallel, each nucleic acid sample having the opportunity to hybridize with an oligonucleotide from each column. As a result, one can, for example, identify one or more microorganisms in a source or determine the identity of a microorganism and the presence of one or more antibiotic or virulence markers present in the microorganism.

The first or second nucleic acid sample can be, or can include, DNA or RNA, and the arrayed detection oligonucleotides can be designed to detect either type of nucleic acid. The DNA and/or RNA detection oligonucleotides can include natural and non-natural nucleotides (e.g., any combination of uracil, adenine, thymine, cytosine and guanine, as well as other bases such as inosine, xanthine, and hypoxanthine).

A source (e.g., a first or second source) for use in the present methods can be virtually any source. While we are concerned with diagnostic methods, the invention is not so limited. Samples obtained from environmental, industrial, or other non-biological settings (e.g., inanimate or non-living sources) can also be tested in the present methods. The industrial source may be a manufacturing or processing plant for food, pharmaceuticals, cosmetics, neutraceuticals, biologics, and the like. Thus, while a source can be derived from an organism (e.g., from a subject such as a human patient), the source can also be an artificial environment (e.g., a laboratory specimen, culture, or surface). In many cases, a source is one that contains or is suspected of containing one or more microorganisms (e.g., bacteria, fungus (e.g., yeast), protozoa, or virus) and, in many instances, those microorganisms will be unwanted in the tested source.

Moreover, the content of the source can be wholly or partially known or unknown. A source can contain, e.g., one or more known microorganisms or one or more microbial nucleic acids in a known or unknown quantity. For example, the source can contain one or more microorganisms (e.g., multiple different bacterial, fungal, and/or protozoal species) at a concentration of less than about 1,000 colony forming units (cfu) per milliliter (ml). Where, e.g., the source contains one or more viral microorganisms, the source can contain at least one virus at a concentration of less than about 1,000 plaque forming units (pfu) per ml. A source can contain any type of microorganism (e.g., any of the microorganisms described herein).

A source can contain two or more (e.g., two, three, four, five, six, seven, eight, nine, 10, 11, 12, 13, 14, 15, 17, 18, 19, 20, 21, 22, 23, 24, 25, or 30 or more) different microorganisms of interest. For example, a source can contain one or more bacteria and fungi, bacteria and virus, bacteria and protozoa, fungi and virus, or any combination thereof. A source can contain two or more different species of interest of the same genus of microorganism. For example, a source can contain two different Candida species (e.g., Candida albicans and Candida glabrata) and/or two different species of Staphylococcus (e.g., Staphylococcus haemolyticus and Staphylococcus aureus). A source can also contain two or more different strains of interest of the same microbial species, e.g., two different E. coli strains.

As noted, virtually any material can be assayed using the methods described herein. In addition to biological samples obtained from, for example, plant or animal matter, a source can be a food sample, a water sample, an air sample, or a commercial product. For example, a source can be, or be obtained from, a food product or water product suspected of being contaminated by a microorganism (e.g., a well water sample or a sample of vegetable produce (e.g., spinach or scallions) suspected of being contaminated with a bacterium such as E. coli or a virus such as hepatitis). In the case of airborne contamination, or the suspicion of airborne contamination, a source can be a sample collected in an air vent or room of a building so suspected of being contaminated with a microorganism such as Legionella pneumophila or Streptococcus pneumoniae.

A source can be a biological sample. Suitable biological samples for the methods described herein include any biological fluid, cell, tissue, or fraction thereof, which includes one or more microorganisms or biomolecules (e.g., microbial DNA or RNA) of interest. A biological sample can be, for example, a specimen obtained from a subject (e.g., a bird, an insect, a reptile, a fish, or mammal (e.g., a rat, mouse, gerbil, hamster, cat, dog, goat, pig, cow, bat, raccoon, horse, non-human primate, or a human)) or can be derived from such a subject. For example, a biological sample can be a tissue section obtained by biopsy, or cells that are placed in or adapted to tissue culture. A biological sample can also be, or include, a biological fluid such as urine, blood, plasma, serum, stool, saliva, milk, sweat, semen, cerebral spinal fluid, tears, wound exudates, skin scrapings, or mucus or mucosal scraping, or such a sample absorbed onto a paper or polymer substrate. A biological sample can be, or include, a pulmonary sample such as, e.g., a sputum sample, a broncheolar lavage sample, an endotracheal aspirate sample, an upper-respiratory mucosal swab, or a protected specimen brush sample. A biological sample can be further fractionated, if desired, to a fraction containing particular cell types. For example, a blood sample can be fractionated into serum or into fractions containing particular types of blood cells such as red blood cells or white blood cells (leukocytes). In some embodiments, two or more sources (e.g., the first and second source) can be different types of samples from the same source or subject. For example, two or more different biological samples such as blood, mucous, sputum, urine, stool, sweat, cerebral-spinal fluid, tears, and/or semen can be obtained from the same subject and analyzed using the methods described herein.

In some embodiments, the methods can include the step of obtaining a biological sample from a subject (e.g., a mammal such as a human) or a non-biological sample from another source. The subject can have, be suspected of having, or be at risk of developing, an infection by any microorganism described herein. Methods for obtaining a biological sample include, e.g., phlebotomy, swab (e.g., buccal swab or drag swab), fine needle aspirate biopsy procedure, broncheolar lavage, endotracheal aspirate, or a protected specimen brush. Biological samples can also be collected, e.g., by microdissection (e.g., laser capture microdissection (LCM) or laser microdissection (LMD)), bladder wash, smear (PAP smear), urine collection, or ductal lavage.

In some embodiments, the methods can include the step of extracting the first nucleic acid sample from a source and/or the second nucleic acid sample from the source. Methods for extracting nucleic acid from a biological sample vary, in part, based on the nature of the nucleic acid (e.g., microbial DNA or microbial RNA) being extracted. For example, DNA can be extracted from a sample by, e.g., contacting the sample with a lysis buffer including one or more detergents (e.g., saponin, sodium dodecyl sulfate, deoxycholine, NP-40, Tween-20, or Triton X-100). In some embodiments, the extraction can also involve mechanical disruption. For example, a mixture of particles (e.g., glass beads) can be added to the sample along with the lysis buffer to aid in disrupting cell membranes (e.g., by vortexing or other shearing techniques). The lysis buffer can also include one or more proteases (e.g., proteinase K) and an RNAase. Following lysis, the extraction process can include precipitating the isolated DNA using, e.g., cold alcohol (e.g., ethanol), a salt (e.g., sodium or potassium acetate), and optionally a carrier such as glycogen. Methods for extracting RNA from a sample are similar to those described above for DNA and can include contacting the sample with a lysis buffer including one or more detergents, RNase-free DNase and RNase-free proteases (as above). RNA isolation can include treating the source and/or any of the buffers or reagents used in the extraction with one or more RNase inhibitors (such as diethylpyrocarbonate (DEPC)) and maintaining the RNA at a neutral or non-basic pH.

In some embodiments, none of the sources (e.g., the first nor the second source) is subjected to any process that would promote propagation of the microorganism. That is, prior to extraction, a source is not subjected to any process that would promote propagation or expansion (through microbial cell division or viral reproduction) of one or more microorganisms suspected of being present in the source. Examples of such a process include, e.g., culturing of the source and/or in embodiments where the source contains (or is suspected of containing) a virus, contacting a population of cultured cells (e.g., host cells) with the source. In some embodiments, nucleic acid is extracted from a source within at least about 24 hours after obtaining the source. Nucleic acid can be extracted from a source less than 60 minutes after obtaining the source. In embodiments where the samples contain (or are suspected of containing) one or more fungi, the first or second nucleic acid sample can be extracted from a source without first isolating any of the fungi from the source.

In some embodiments, the step of amplifying (if a selected region of nucleic acid sequence is present) at least one selected region of nucleic acid sequence in each of the first and second nucleic acid samples can be performed under conditions that permit detection of the amplified first or second nucleic acid sequence if the concentration of the microorganism exceeds a threshold concentration. The conditions can include varying, e.g., the number of PCR cycles used to amplify the selected region(s), the amount of nucleic acid sample used for amplification, the temperature at which the amplification and/or annealing step is performed, the extension time, and/or the concentration of primers added to the amplification reaction).

In some embodiments, the step of contacting the array can be performed under conditions that permit detection of the amplified first or second nucleic acid sequence if the concentration of the microorganism exceeds a threshold concentration. The conditions can include, e.g., varying: (i) the amount of amplified first or second nucleic acid contacted with the array; (ii) the temperature at which the first or second nucleic acid is contacted with the array; or (iii) the concentration or binding efficiency of the detection oligonucleotides on the porous solid support.

The threshold concentration can be, e.g., at least or about 10², 10³, 10⁴, or between about 10⁵-10⁶ cfu/mL. The threshold can depend on, e.g., the type of source. For example, the threshold can be about 10³ cfu/mL for a protected specimen brush sample, about 10⁴ cfu/mL for a bronchoalveolar lavage sample, or between about 10⁵-10⁶ cfu/mL for a endotracheal aspirate sample.

In some embodiments, the selected region of nucleic acid sequence can be, or contain, at least a portion of a gene conferring antibiotic resistance or virulence. The gene conferring antibiotic resistance can be a gene encoding a β-lactamase (e.g., a carbapenemase). The gene conferring virulence can be a gene encoding a bacterial toxin. In some embodiments, the selected region of nucleic acid sequence can be, or contain, at least a portion of a gene conferring a pathogenic characteristic to the microorganism. For example, the pathogenic characteristic could be increased growth, resistance or increased resistance to a toxic environment (e.g., high or low pH, high or low temperature, radiation, or heavy metals), or an increased metabolism.

In some embodiments, the selected region of nucleic acid sequence can be, or contain, at least a portion of (or all or part of) a polymorphic region. The polymorphic region can be, e.g., a hypervariable region of a ribosomal DNA (rDNA) or ribosomal RNA (rRNA) from a microorganism. The selected region of nucleic acid sequence can be, or contain, at least a portion of (or all or part of) a gene encoding a large subunit of microbial rRNA or a gene encoding a small subunit of a microbial rRNA. The large subunit can be encoded by, e.g., a 23S rDNA, a 25S rDNA, a 26S rDNA, or a 28S rDNA gene. The small subunit can be encoded by, e.g., a 16S or 18S rDNA. The polymorphic region can be, or contain, all or part of a gene encoding a β-lactamase.

In some embodiments, the amplified first or second nucleic acids can be detectably labeled. The nucleic acids can be detectably labeled during amplification or following amplification. For example, an PCR or reverse transcription-PCR (RT-PCR) amplification step can be used to detectably-label the amplified nucleic acid, e.g., using detectably labeled primers. Alternatively, the amplified nucleic acids can be labeled during amplification by using detectably labeled nucleotides (e.g., nucleotide analogues or radiolabeled nucleotides). A detectable label can be enzymatically (e.g., by nick-translation or kinase (e.g., T4 polynucleotide kinase)) or chemically conjugated to the amplified nucleic acid following amplification. Detectable labels include, e.g., fluorescent labels (e.g., umbelliferone, fluorescein, fluorescein isothiocyanate, rhodamine, dichlorotriazinylamine fluorescein, dansyl chloride, allophycocyanin (APC), or phycoerythrin); luminescent labels (e.g., europium, terbium, or Qdot™ nanoparticles); radionuclide labels (e.g., ¹²⁵I, ¹³¹I, ³⁵S, ³²P, ³³P, or ³H); chemical labels; a label that recruits an enzyme; or labels detectable by an antibody or ligand-binding proteins specific for the detectable label (e.g., digoxigenin and biotin).

In some embodiments, the array can contain at least one detection oligonucleotide comprising a detectable label as a positive control; as an “always detectable signal.” In some embodiments, the array contains at least one detection oligonucleotide that hybridizes with an amplified first or second nucleic acid to identify a Gram-positive bacterium. In some embodiments, the array contain at least one detection oligonucleotide that hybridizes with an amplified first or second nucleic acid to identify a Gram-negative bacterium. In some embodiments, hybridization of at least one detection oligonucleotide to at least one of the amplified first or second nucleic acids identifies a microorganism as being a Gram-positive bacterium. In some embodiments, hybridization of at least one detection oligonucleotide to at least one of the amplified first or second nucleic acids identifies a microorganism as a Gram-negative bacterium.

In some embodiments, the array can contain one or more (e.g., two, three, four, five, six, seven, eight, nine, 10, 11, 12, 13, 14, 15, 16, 17, 18, 19, 20, 21, 22, 23, 24, 25, 26, 27, 28, 29, 30, 31, or 32 or more) detection oligonucleotides containing, or consisting of, one or more of any one of SEQ ID NOS:1-32.

In some embodiments, the porous solid support can be a membrane such as a nitrocellulose or nylon membrane. The porous solid support can be in a cassette and used, e.g., in conjunction with a flow-through device.

In embodiments where a flow-through device is used, a solution containing the nucleic acids can be contacted with the array and subsequently passed through the array by, e.g., means of a vacuum applied to the flow-through device. The flow-through device can be configured such that the contacting occurs with or without agitation. The device can also be configured such that the rate at which the solution containing the nucleic acids, or subsequent wash solutions or detection solutions, is passed through the porous solid support can be adjusted. The cassette of the flow-through device can be configured such that a first and second amplified nucleic acid sample (or three or more (e.g., three, four, five, six, seven, eight, nine, 10, 11, 12, or 15 or more) nucleic acid samples) can be contacted, in parallel, to an array within the cassette without mixing of one nucleic acid sample with another on the array. For example, the cassette can have channels or physical barriers between different sections (different sets of oligonucleotides) of the array.

Use of a flow-through device in conjunction with any of the methods described herein may have several advantages. For example, use of a flow-through device can increase the speed at which the methods can be performed without sacrificing sensitivity of detection or false-positive or -negative rates. The flow-through device also allows for easy sequestration of waste products, e.g., biohazardous or radioactive waste products.

In some embodiments, the method can include the step of, after identifying one or more microorganisms in one or more sources, creating a record indicating that one or more microorganisms are present in the one or more sources. The record can be on a computer-readable medium.

In embodiments where one or more microorganisms are identified in a biological sample from a subject, the method can also include the step of detecting genes encoding antibiotic resistance or virulence factors. This can aid in selecting an appropriate anti-microbial therapeutic regimen (e.g., an antibiotic, an anti-fungal, an anti-viral, or anti-protozoal agent) for a subject. In embodiments where the method is used to both identify a microorganism and determine the presence of one of more antibiotic resistance genes in the microorganism, the selection can involve choosing an appropriate therapy to which the microorganism is not resistant. Selecting a therapy for a subject can be, e.g.: (i) writing a prescription for a medicament; (ii) giving (but not necessarily administering) a medicament to a subject (e.g., handing a sample of a prescription medication to a patient while the patient is at the physician's office); (iii) communication (verbal, written (other than a prescription), or electronic (email, post to a secure site)) to the patient of the suggested or recommended anti-microbial treatment regimen (e.g., an antibiotic); or (iv) identifying a suitable anti-microbial treatment regimen for a subject and disseminating the information to other medical personnel, e.g., by way of patient record. The latter (iv) can be useful in a case where, e.g., more than one therapeutic agent are to be administered to a patient by different medical practitioners.

After selecting an appropriate anti-microbial treatment regimen for an infected subject, a medical practitioner (e.g., a doctor, physician's assistant, nurse, or the like) can administer the appropriate anti-microbial treatment regimen (e.g., a regimen comprising one or more anti-microbial agents) to the subject. In other embodiments, the anti-microbial treatment regimen can be administered by someone other than a medical practitioner (e.g., the anti-microbial treatment regimen can be self administered). Suitable anti-microbial therapeutic agents (e.g., antibacterial agents, anti-fungal agents, or anti-viral agents) include, e.g., aminoglycosides (e.g., amikacin, gentamicin, kanamycin, neomycin, netilmicin, streptomycin, or tobramycin); ansamycins, cephalosporins (e.g., cefaclor, cefamandole, cefoxitin, or cefprozil); macrolides (e.g., azithromycin, clarithromycin, erthyromycin, or roxithromycin); penicillins (e.g., amoxicillin, ampicillin, azlocillin, carbenicillin, penicillin, piperacillin, or ticarcillin); quinalones (e.g., ciprofloxacin, enoxacin, levofloxacin, ofloxacin, or moxifloxacin); tetracyclines (e.g., doxycycline, micocycline, or tetracycline); imidazoles (e.g., miconazole, ketoconazole, clotrimazole, econazole, bifonazole, butoconazole, or fenticonazole); triazoles (e.g., fluconazole, itraconazole, isavuconazole, ravuconazole, posaconazole, voriconazole, or terconazole); anti-virals (e.g., abacavir, acyclovir, acyclovir, adefovir, amantadine, amprenavir, arbidol, atazanavir, atripla, ganciclovir, gardasil, indinavir, inosine, integrase inhibitor, interferon, nucleoside analogues, penciclovir, protease inhibitors, reverse transcriptase inhibitors, or saquinavir); and anti-protozoals including nitazoxanide, metronidazole, eflornithine, furazolidone, hydroxychloroquine, iodoquinol, and pentamidine.

In yet another aspect, the invention features a method for selecting an anti-microbial therapeutic regimen for a subject, the method comprising: identifying, in parallel: (i) a microorganism in a biological sample obtained from a subject and (ii) the presence of an antibiotic resistance marker (e.g., an antibiotic resistance gene such as any of the antibiotic resistance genes described herein); and selecting an anti-microbial therapeutic regimen that is effective to reduce or eliminate an infection by the antibiotic resistant microorganism.

In another aspect, the invention features an isolated polynucleotide sequence consisting of any one of SEQ ID NOS:1-32 or 34-37 or a sequence complementary thereto or a functionally active variant at least 80% identical to any one of SEQ ID NOs:1-32 or 34-37 or a sequence complementary thereto. The polynucleotide sequences can further include a heterologous nucleotide sequence.

In another aspect, the invention features an isolated polynucleotide sequence consisting of any of the nucleotide sequences described herein or a sequence complementary thereto or a functionally active variant at least 80% identical to (e.g., 85%, 90%, or 95% identical to) any one of the nucleic acid sequences described herein or a sequence complementary thereto. The polynucleotide sequences can further include a heterologous nucleotide sequence.

In another aspect, the invention features a composition comprising a plurality of polynucleotides. The composition can be immobilized, e.g., on a solid support. Each detection oligonucleotide, or at least one different oligonucleotide, in the plurality can be immobilized at predetermined positions such that each detection oligonucleotide can be identified by its position.

The plurality can contain at least two (e.g., two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, or 35 or more) detection oligonucleotides comprising, or consisting of, any one of SEQ ID NOS:1-32, a sequence complementary thereto, or a functionally active variant at least 80% identical to any one of SEQ ID NOs:1-32, or a sequence complementary thereto.

The plurality can contain at least two (e.g., two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, or 35 or more) detection oligonucleotides comprising, or consisting of, any of the nucleic acid sequences described herein, a sequence complementary thereto, or a functionally active variant at least 80% identical to any one of the nucleic acid sequences described herein, or a sequence complementary thereto.

The polynucleotide arrays can be attached to a solid support, e.g., a porous or non-porous material that is insoluble. The polynucleotides can be associated with the support in variety of ways, e.g., they can be covalently or non-covalently bound.

A support can be composed of a natural or synthetic material or an organic or inorganic material. The composition of the solid support on which the polynucleotide sequences are attached (either 5′ or 3′ terminal attachment) generally depends on the method of attachment (e.g., covalent attachment). Suitable solid supports include, but are not limited to, plastics, resins, polysaccharides, silica or silica-based materials, functionalized glass, modified silicon, carbon, metals, inorganic glasses, membranes, nylon, natural fibers such as silk, wool and cotton, or polymers. A porous solid support can be, or include, a membrane such as nitrocellulose or a nylon membrane. The material comprising the solid support can have reactive groups such as carboxy, amino, or hydroxyl groups, which are used for attachment of the polynucleotides. Polymeric solid supports can include, e.g., polystyrene, polyethylene glycol tetraphthalate, polyvinyl acetate, polyvinyl chloride, polyvinyl pyrrolidone, polyacrylonitrile, polymethyl methacrylate, polytetrafluoroethylene, butyl rubber, styrenebutadiene rubber, natural rubber, polyethylene, polypropylene, (poly)tetrafluoroethylene, (poly)vinylidenefluoride, polycarbonate, or polymethylpentene).

In some embodiments, the solid support is a particle, e.g., an encoded particle. Each particle includes a unique code (such as a bar code, luminescence code, fluorescence code, a nucleic acid code, and the like). Encoding can be used to provide particles for evaluating different nucleic acids in a single biological sample. The code is embedded (for example, within the interior of the particle) or otherwise attached to the particle in a manner that is stable through hybridization and analysis. The code can be provided by any detectable means, such as by holographic encoding, by a fluorescence property, color, shape, size, weight, light emission, quantum dot emission and the like to identify the particle and thus the capture detection oligos immobilized thereto. Encoding can also be accomplished by varying a ratio of two or more dyes in one particle; the ratio in one particle would be different from the ratio present in another particle. For example, the particles may be encoded using optical, chemical, physical, or electronic tags. Examples of such coding technologies are optical bar codes fluorescent dyes, or other means. In some embodiments, the particle code is a nucleic acid, e.g., a single stranded nucleic acid.

The compositions can also include one or more control detection oligonucleotides. For example, the plurality can contain at least one non-specific detection oligonucleotide that will not specifically hybridize to any of the amplified nucleic acids. The non-specific oligonucleotide can have, e.g., the sequence TTTTTTTTTTTTTTTTTTTT (SEQ ID NO:33). The non-specific oligonucleotide can be detectably labeled, e.g., a digoxigenin labeled non-specific oligonucleotide. The polynucleotide arrays can also include one or more positive control detection oligonucleotides. For example, an array can contain one or more detection oligonucleotides that specifically hybridize with an amplified nucleic acid known to be present in a sample.

In yet another aspect, the invention features a kit comprising any of the compositions described above and instructions for use. The kit can include, e.g., a broad-range primer set. The broad-range primer set can binds to a region of DNA that is present in more than one bacterial microorganism or more than one fungal microorganism. The broad-range primer set can include, e.g., primers containing, or consisting of, any of SEQ ID NO:38 or SEQ ID NO:39 or SEQ ID NOs:34-37.

In yet another aspect, the invention features an isolated fungal cell. The cell contains an exogenous nucleic acid sequence flanked at both the 5′ and 3′ ends by a nucleic acid sequence, which encodes all or part of a bacterial 23S rRNA. The exogenous nucleic acid sequence can be autonomously replicating or can be integrated into the genome of the fungal cell. The exogenous nucleic acid sequence can contain all or part of a bacterial NodA gene (e.g., a rhizobial bacterial NodA gene). The bacterial NodA gene can be a S. meliloti NodA gene. The fungal cell can be a mould or a yeast. The yeast can be, e.g., S. cerevisiae or any other yeast described herein.

In some embodiments, the nucleic acid sequences encoding the bacterial 23S rRNA can contain a sequence that hybridizes to SEQ ID NO:38 or SEQ ID NO:39.

When the DNA from the fungal cell containing the NodA gene is subject to amplification with the appropriate 23S primers (e.g., SEQ ID NO:38 or SEQ ID NO:39), the amplification product can be detectable by NodA-specific probes on the array. Because the cell that provides the DNA is fungal, no bacterial genomic DNA (except for the 5′ and 3′ flanking primer-binding sequences and the NodA gene itself) is amplified. The NodA gene produced by the amplification is not expected to be found in human pathogens. Therefore the fungal cell can function as a positive control that produces a unique signal on the blot if the assay is successfully performed.

In another aspect, the invention features an isolated fungal cell containing a vector. The vector contains all of part of a bacterial NodA gene such as a rhizobial bacterial NodA gene (e.g., a S. meliloti NodA gene). The vector can be, e.g., a plasmid, a yeast artificial chromosome, a viral vector, or a retrotranspon). The NodA gene can be, e.g., flanked both at the 5′ and 3′ end by a nucleic acid sequence encoding all or part of a bacterial 23S rRNA. In some embodiments, the nucleic acid sequences encoding the bacterial 23S rRNA can contain a sequence that hybridizes to SEQ ID NO:38 or SEQ ID NO:39.

In yet another aspect, the invention features a kit comprising any of the isolated fungal cells described herein and, optionally, instructions for extracting nucleic acid from the cell.

Any of the methods and compositions described herein can be used to identify a variety of microorganisms including, e.g., bacteria, fungus (e.g., yeast), protozoa, and virus. Examples of bacteria (e.g., Gram-negative or Gram-positive bacteria) that can be detected include, but are not limited to, a species of a genus Staphylococcus, Streptococcus, Enterococcus, Escherichia, Citrobacter, Helicobacter, Enterobacter, Haemophilus, Pseudomonas, Serratia, Stenotrophomonas, Proteus, or Legionella. The bacterium can be, e.g., Staphylococcus epidermidis, Staphylococcus warneri, Staphylococcus saprophyticus, Staphylococcus xylosus, Staphylococcus cohnii, Staphylococcus simulans, Staphylococcus hominus, Staphylococcus haemolyticus, Staphylococcus aureus, Streptococcus milleri, Streptococcus pneumoniae, Streptococcus spp., Streptococcus bovis, Streptococcus pyogenes, Streptococcus. agalactiae, Streptococcus. anginosus, Streptococcus. mutans, Streptococcus. oralis, Streptococcus. salivarius, Enterococcus faecium, Enterococcus faecalis, Escherichia coli, Klebsiella oxytoca, Klebsiella pneumoniae, Enterobacter cloaeae, Enterobacter aerogenes, Citrobacter freundii, Proteus mirabilis, Serratia marcescens, Pseudomonas aeruginosa, Stenotrophomonas maltophilia, Legionella pneumophila, Acinetobacter baumannii, or Burkholderia cepacia. Examples of fungus include, e.g., moulds and yeasts. A yeast can be a species of a genus of yeast selected from the group consisting of Candida, Cryptococcus, Histoplasma, and Exophiala. Yeasts include, e.g., Candida albicans, Candida glabrata, Candida kruzei, Candida parapsilosis, Candida tropicalis, Aspergillus fumigatus, Cryptococcus neoformans, or Pneumocystis carinii. Protozoa (e.g., infectious protozoa) include, e.g., Entamoeba histolytica, Giardia lamblia, Trypanosoma brucei, Toxoplasma gondii, or species of the genus Plasodium. Examples of viruses that can be identified using the methods and compositions described herein include, e.g., herpes simplex viruses (HSV), retroviruses (e.g., human immunodeficiency virus (e.g., HIV-1)), hepatitis viruses (e.g., hepatitis A, B, or C), enteroviruses, papillomaviruses (e.g., HPV), Epstein-Barr virus (EBV), rotaviruses, cytomegaloviruses, influenza viruses, or pox viruses.

A subject (e.g., a human patient) having an infection can be one with any of a variety of types of infection (microbial infections) such as a bacterial, fungal, protozoal, or viral infection. Bacterial infections include, e.g., colitis, endocarditis, meningitis, pneumonia, osteomyelitis, otitis media, cutaneous ulcers (e.g., decubitis ulcers), urinary tract infections, or bacteremias such as sepsis, septic joint, septic shock, toxic shock syndrome, or disseminated intravascular coagulation. Fungal infections can include, but are not limited to, fungemia, aspergillosis, blastomycosis, candidiasis, coccidioidomycosis, cryptococcosis, fungal infections of fingernails or toenails, fungal sinusitis, histoplasmosis, hypersensitivity pneumonitis, mucormycosis, paracoccidioidomycosis, or sporotrichosis. Viral infections include e.g., HIV infection, influenza, viral meningitis, vial hepatitis, SARS, herpes and viral penumonia. Protozoal infections include e.g., amebiasis, babesiosis, coccidiosis, cryptosporidiosis, giardiasis, leishmaniasis, malaria, protozoal meningoencephalitis, toxoplasmosis, or trypanosomiasis.

A subject having an infection can suffer from anthrax infections, bronchitis, Bubonic plague, Cat-Scratch Fever, cellulitis, chickenpox, Chlamydia, croup, Dengue fever, ebola, encephalitis, keratitis, Fifth's disease, flu, folliculitis, genital warts, gum disease, syphilis, Chlamydia, Hand-Foot-Mouth disease, hot tub rash, kidney infections, laryngitis, leprosy, Lyme disease, measles, monkeypox, mononucleosis, necrotizing fasciitis, pink eye, pneumonia, ring worm, Rocky mountain fever, rubella, scarlet fever, smallpox, thrush, West Nile infection, or whooping cough.

All nucleotide sequences described herein (e.g., polynucleotide sequences depicted in Tables 1 and 2) are presented in standard IUB/IUPAC conventional nucleic acid code. For example, A (adenosine), C (cytidine), G (guanine), T (thymidine), U (uridine), R (guanine or adenosine), Y (thymidine or cytidine), and K (guanine or thymidine).

Other features and advantages of the methods and compositions will be apparent from the description below, from the drawings, and from the claims.

### BRIEF DESCRIPTION OF THE DRAWINGS

FIG. 1 is a photograph of a hybridized polynucleotide blot depicting the detection of antibiotic resistance markers in Klebsiella pneumonia (KPC-1 carbapenemase or KPC-2 carbapenemase) and Serratia marcescens (SME carbapenemase). “Primers” denote the primer set used to amplify DNA extracted from bacterial cultures containing the various bacteria. “Strains” indicate the various Klesiella and Serratia strains carrying the antibiotic resistance markers. Specific detection polynucleotides are indicated at the left of the blot (rows 3-25) and control rows “ink” and “buffer” are also indicated.

FIG. 2 is a photograph of a hybridized polynucleotide blot depicting the detection of antibiotic resistance markers in E. coli strains containing TEM β-lactamase alleles TEM-10 or TEM-26. E. coli not containing the TEM markers, S. aureus, and non-infected blood were used as negative controls. Specific detection polynucleotides are indicated at the left of the blot (rows 4-17) and control rows “ink” and “buffer” are also indicated. “10 ul” and “1 ul” refer to the amount of extracted DNA in each set of detected lanes.

FIG. 3 is a photograph of a hybridized polynucleotide blot depicting the detection of toxigenic markers in the C. difficile NAP2 strain containing toxin A and toxin B. “Primers” denote the primer set used to amplify DNA extracted from bacterial cultures containing the various bacteria. “Strains” indicate the various C. difficile strains (toxigenic and non-toxigenic) and C. perfringens (as a control) analyzed in the methods. Specific detection polynucleotides are indicated at the left of the blot (rows 3-21) and control rows “ink” and “buffer” are also indicated.

### DETAILED DESCRIPTION

The invention features, inter alia, methods and compositions for identifying one or more microorganisms in one or more samples, e.g., by detecting the presence of one or more nucleic acids that so identify the microorganisms. Such methods and compositions can be useful in, e.g., determining whether a subject has a microbial infection (e.g., bacterial, viral, fungal, or parasitic infection), and the causative microorganism underlying the infection. The compositions and methods are also be useful in detecting the presence of microbial genetic elements (e.g., on the chromosomes or plasmids, acquired or endogenous) conferring antibiotic resistance or virulence factors. Any or all of these can applications be useful in determining an appropriate therapeutic modality (e.g., an appropriate anti-fungal or antibiotic) for a subject identified as having an infection.

Arrays and Kits: The arrays, and kits containing the arrays, described herein are useful in, e.g., detecting the presence of one or more nucleic acids (e.g., microbial DNA or RNA) in a sample and thus, identifying one or more microorganisms in one or more samples. The kits and compositions are also useful for diagnosing a subject as having an infection and/or selecting an appropriate therapeutic modality for a subject having, suspected of having, or at risk of developing an infection.

The arrays can include at least two (e.g., two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, or 35 or more) detection oligonucleotides comprising (or consisting of) all or a fragment of any of the nucleotide sequences depicted in Table 1).

TABLE 1

| | Corresponding | SEQ |
|---|---|---|
| Nucleotide Sequence | Organism | ID NO: |
| GAATACATAGGTTAACGAGGCGAA | Klebsiella | 1 |
| GCGTCTGGAAAGTCGCAG | Klebsiella | 2 |
| TACATAGCATATCAGAAGGCACACC | S. aureus | 3 |
| GGACTGCGATATAGGATTAATCATTAT | S. agalactiae | 4 |
| CCCATTAAGTTATGTGTGTTTTAGTGG | Acinetobacter | 5 |
| | baumannii | |
| AGYTTRCTYXTYGGGGTTGTAGGAC | Universal | 6 |
| | Gram-positive | |
| GGAAAAGAAATCAACCGAGATTC | Universal | 7 |
| | Gram-negative | |
| GGTATCGTAATTGAAGAGGTTTGG | C. difficile | 8 |
| GGTGGGAAACTGGAGCAGTTCC | C. difficile, | 9 |
| | toxin A | |
| TTCAATTCTGATGGAGTTATGCA | C. difficile, | 10 |
| | toxin B | |
| TTGCATGCTGCTCTCTCGG | Candida | 11 |
| TAGGATAAGTGCAAAGAAATGTGGC | Candida | 12 |
| GAATTGCGTTGGAATGTGGCA | Candida | 13 |
| TGCAGGAGAAGGGGTTCTGG | Candida | 14 |
| CGATACTTGTTATCTAGGATGCTGG | Candida | 15 |
| CACCGTCATGCCTGTTGTCAG | KPC gene | 16 |
| ACCTAATGTCATACCTGAGCCTTT | SME gene | 17 |
| GCAGCAGAAGCCATATCACCTAAT | SME gene | 18 |
| ACTGCGTTGTGGGACGACA | Streptococcus | 19 |
| ACTGCGACGTGGGACTTTAAAA | Streptococcus | 20 |
| AAAGCAGCCAAGGGAATAGAAG | Streptococcus | 21 |
| AGGTACTACCTGTTACCCGCATC | Streptococcus | 22 |
| GTAATACCTGTTACCCACATCTGTT | Streptococcus | 23 |
| CGAAACGGCAGGAGGGCAAACC | Streptococcus | 24 |
| ATGAGAAGGAAGACGCAGTGAA | Streptococcus | 25 |
| ACGTGGGACTTTAAAAGGATAGAA | Streptococcus | 26 |
| AAGAGCCTCGTATTTGAAATTCAC | Streptococcus | 27 |
| GCGATTGCCTTAGTAGCGG | Streptococcus | 28 |
| AGCGAAACGGCAGGAGGG | Streptococcus | 29 |
| CTTAATGAAACGGCGCAACACG | IC | 30 |
| CCAACTGCGGCCACCCTCAAAT | IC | 31 |
| GCGTGGAAGGGAGATCGGCGTT | IC | 32 |

IC refers to “internal control.”

Fragments of any of the oligonucleotides described herein (e.g., any of the nucleotide sequences depicted in Table 1 or Table 2) can include at least five (e.g., at least six, at least seven, at least eight, at least nine, at least 10, at least 11, at least 12, at least 13, at least 14, at least 15, at least 20, at least 25, at least 30 or more) nucleotides of the full-length sequence (e.g., a full-length sequence depicted in any of SEQ ID NOS: 1-32 and 34-37).

A oligonucleotide can consist of, or contain, all or an active fragment of any of the polynucleotide sequences described herein (e.g., a oligonucleotide sequence depicted in Table 1 or Table 2) and, optionally, additional heterologous nucleotide sequence(s) flanking the oligonucleotide. For example, an oligonucleotide (e.g., any one of SEQ ID NOS:1-32 and 34-37) can have one or more (e.g., two or more, three or more, four or more, five or more, six or more, seven or more, eight or more, nine or more, 10 or more, 11 or more, 12 or more 15 or more, 20 or more, 25 or more, 30 or more, 35 or more, or 40 or more) additional heterologous nucleotides on the 5′ end, the 3′ end, or the 5′ and 3′ end. In some embodiments, the additional heterologous nucleotide sequence can be longer than the base polynucleotide sequence to which it is attached. Accordingly, as used herein, an “oligonucleotide comprising” a particular nucleotide sequence (e.g., an oligonucleotide depicted in Table 1 or Table 2) refers to a sequence comprising: (i) a nucleic acid sequence consisting of any polynucleotide (e.g., any oligonucleotide sequence depicted in Table 1 or Table 2) and, optionally, (ii) a heterologous nucleotide sequence.

An oligonucleotide can also contain, or consist of a nucleic acid sequence that is complementary to all or a fragment of any of the nucleotide sequences depicted in Table 1 or 2. An oligonucleotide can also contain, or consist of, a nucleic acid sequence that is at least 70 (e.g., at least 72, 75, 77, 80, 82, 85, 87, 90, 92, 95, 97, or 98) % identical to all or a fragment of a nucleotide sequence depicted in Table 1 or 2.

The polynucleotide can be single or double-stranded (e.g., a nucleic acid sequence depicted in Table 1 hybridized to its corresponding complementary sequence) and of variable length. In some embodiments, the length of one strand of a polynucleotide (e.g., a polynucleotide sequence comprising all or a fragment of a nucleotide sequence in Table 1) can be about five nucleotides (e.g., about five nucleotides, about seven nucleotides, about eight nucleotides, about nine nucleotides, about 10 nucleotides, about 12 nucleotides, about 13 nucleotides, about 14 nucleotides, about 15 nucleotides, about 20 nucleotides, about 25 nucleotides, about 30 nucleotides, about 35 nucleotides, about 40 nucleotides, about 50 nucleotides, about 75 nucleotides, about 100 nucleotides, or about 150 or more nucleotides). A longer polynucleotide often allows for higher stringency hybridization and wash conditions. The polynucleotide can be DNA, RNA, modified DNA or RNA, or a hybrid where the nucleic acid contains any combination of the foregoing, and any combination of uracil, adenine, thymine, cytosine and guanine, as well as other bases such as inosine, xanthine, and hypoxanthine.

In some embodiments, an array can have one or more (e.g., two or more, three or more, four or more, five or more, six or more, seven or more, eight or more, nine or more, 10 or more, 11 or more, 12 or more, 13 or more 14 or more, 15 or more, 16 or more, 17 or more, 18 or more, 19 or more, 20 or more, 21 or more, 22 or more, or all 23) of the oligonucleotides (or fragments thereof) depicted in Table 1 and one or more additional oligonucleotide such as those described in, e.g., PCT Publication No. WO 00/052203 and Anthony et al. (2000) J. Clin. Microb. 38:781-788, the disclosures of each of which are incorporated herein by reference in their entirety. The oligonucleotides can be attached to a solid support, e.g., a porous or non-porous material that is insoluble. The oligonucleotides can be associated with the support in variety of ways, e.g., covalently or non-covalently bound.

A support can be composed of a natural or synthetic material, an organic or inorganic material. The composition of the solid support on which the oligonucleotides are attached (either 5′ or 3′ terminal attachment) generally depends on the method of attachment (e.g., covalent attachment). Suitable solid supports include, but are not limited to, plastics, resins, polysaccharides, silica or silica-based materials, functionalized glass, modified silicon, carbon, metals, inorganic glasses, membranes, nylon, natural fibers such as silk, wool and cotton, or polymers. The material comprising the solid support can have reactive groups such as carboxy, amino, or hydroxyl groups, which are used for attachment of the oligonucleotides. Polymeric solid supports can include, e.g., polystyrene, polyethylene glycol tetraphthalate, polyvinyl acetate, polyvinyl chloride, polyvinyl pyrrolidone, polyacrylonitrile, polymethyl methacrylate, polytetrafluoroethylene, butyl rubber, styrenebutadiene rubber, natural rubber, polyethylene, polypropylene, (poly)tetrafluoroethylene, (poly)vinylidenefluoride, polycarbonate, or polymethylpentene (see, e.g., U.S. Pat. No. 5,427,779, the disclosure of which is hereby incorporated by reference in its entirety). Alternatively, the oligonucleotide sequences can be attached to the solid support without the use of such functional groups.

Each different oligonucleotide of an array can be immobilized at predetermined positions such that each different oligonucleotide can be identified by its position.

Methods of attaching one or more oligonucleotides to a solid support are known in the art, and are set forth in the accompanying Examples. For example, amino-linked oligonucleotides can be attached to a solid support (such as a membrane (e.g., a Biodyne™ C membrane)) using 1-ethyl-3-dimethylaminopropylcarbodiimide (EDAC)-mediated cross-linking (see, e.g., working Examples, U.S. Pat. No. 5,391,723 and Penchovsky et al. (2000) Nucleic Acids Res. 28(22):e98, the disclosures of each of which are incorporated herein by reference in their entirety).

Additional methods of attaching one or more oligonucleotides to a solid support include, e.g., cross-linking polynucleotides to a membrane using UV-light, photolithography, or chemical cross-linking agents (see, e.g., Kumar et al. (2004) Nucleic Acids Res. 32(10):e80; PCT Publication No. WO 00/052203; and U.S. Pat. Nos. 5,405,783; 5,910,406; and 6,077,674, the disclosures of each of which are incorporated herein by reference in their entirety). Oligonucleotides can also be synthesized directly on a solid support, e.g., a silicon chip, as described in Lu et al. (Proc. SPIE 4224:118-121, 2000) and U.S. Pat. No. 5,733,509, the disclosures of each of which are incorporated by reference in their entirety.

The attachment and proper alignment of the oligonucleotides to a solid support can be aided through the use of, e.g., a miniblotter device (see, e.g., U.S. Pat. Nos. 4,834,946 and 4,713,349, the disclosures of each of which are incorporated herein by reference in their entirety).

The arrays can also be conjugated to solid support particles. Many suitable solid support particles are known in the art and illustratively include, e.g., particles, such as Luminex®-type encoded particles, magnetic particles, and glass particles. Another exemplary platform uses holographic barcodes to identify cylindrical glass particles. For example, Chandler et al. (U.S. Pat. No. 5,981,180) describes a particle-based system in which different particle types are encoded by mixtures of various proportions of two or more fluorescent dyes impregnated into polymer particles. Soini (U.S. Pat. No. 5,028,545) describes a particle-based multiplexed assay system that employs time-resolved fluorescence for particle identification. Fulwyler (U.S. Pat. No. 4,499,052) describes an exemplary method for using particle distinguished by color and/or size. U.S. Publication Nos. 2004-0179267, 2004-0132205, 2004-0130786, 2004-0130761, 2004-0126875, 2004-0125424, and 2004-0075907 describe exemplary particles encoded by holographic barcodes.

U.S. Pat. No. 6,916,661 describes polymeric microparticles that are associated with nanoparticles that have dyes that provide a code for the particles. The polymeric microparticles can have a diameter of less than one millimeter, e.g., a size ranging from about 0.1 to about 1,000 micrometers in diameter, e.g., 3-25 μm or about 6-12 μm. The nanoparticles can have, e.g., a diameter from about 1 nanometer (nm) to about 100,000 nm in diameter, e.g., about 10-1,000 nm or 200-500 nm.

Any of the arrays described herein can also include one or more control detection oligonucleotides. For example, an array can contain a non-specific detection oligonucleotide that will not specifically hybridize to any of the amplified nucleic acids. The non-specific oligonucleotide can have, e.g., the sequence TTTTTTTTTTTTTTTTTTTT (SEQ ID NO:33). The non-specific oligonucleotide can be detectably labeled, e.g., a digoxigenin labeled non-specific oligonucleotide. The polynucleotide arrays can also include one or more positive control oligonucleotides. For example, an array can contain one or more detection oligonucleotides that will specifically hybridize with a microbial nucleic acid known to be present in a sample.

The arrays can have two or more (e.g., three or more; four or more; five or more; six or more; seven or more; eight or more; nine or more; 10 or more; 11 or more; 12 or more; 13 or more 14 or more; 15 or more; 16 or more; 17 or more; 18 or more; 19 or more; 20 or more; 21 or more; 22 or more; 23 or more; 24 or more; 25 or more; 30 or more; 35 or more; 40 or more; 42 or more; 45 or more; 47 or more; 50 or more; 52 or more; 55 or more; 57 or more; 60 or more; 62 or more; 65 or more; 67 or more; 70 or more; 75 or more; 80 or more; 85 or more; 90 or more; 95 or more; 100 or more; 150 or more; 200 or more; 300 or more; 400 or more; 500 or more; 600 or more; 1,000 or more; 2,000 or more; 5,000 or more; 10,000 or more; 20,000 or more; 30,000 or more; 50,000 or more; or 100,000 or more) detection oligonucleotides.

The arrays can have two or more (e.g., three or more; four or more; five or more; six or more; seven or more; eight or more; nine or more; 10 or more; 11 or more; 12 or more; 13 or more 14 or more; 15 or more; 16 or more; 17 or more; 18 or more; 19 or more; 20 or more; 21 or more; 22 or more; 23 or more; 24 or more; 25 or more; 30 or more; 35 or more; 40 or more; 42 or more; 45 or more; 47 or more; 50 or more; 52 or more; 55 or more; 57 or more; 60 or more; 62 or more; 65 or more; 67 or more; 70 or more; 75 or more; 80 or more; 85 or more; 90 or more; 95 or more; 100 or more; 150 or more; 200 or more; 300 or more; 400 or more; 500 or more; 600 or more; 1,000 or more; 2,000 or more; 5,000 or more; 10,000 or more; 20,000 or more; 30,000 or more; 50,000 or more; or 100,000 or more) different detection oligonucleotides.

In some embodiments, the arrays can have less than 100,000 (e.g., less than 90,000; less than 80,000; less than 70,000; less than 60,000; less than 50,000; less than 40,000; less than 30,000; less than 20,000; less than 15,000; less than 10,000; less than 5,000; less than 4,000; less than 3,000; less than 2,000; less than 1,500; less than 1,000; less than 750; less than 500, less than 200, less than 100, less than 90, less than 80, less than 70, less than 60, less than 55, less than 50, less than 45, or less than 40) different detection oligonucleotides.

Also provided are kits containing any of the arrays described herein. The kits can, optionally, contain instructions for identifying one or more microorganisms in a sample.

In some embodiments, the kits can contain two or more different oligonucleotides, a solid support, and instructions for making an array of oligonucleotides bound to the solid support. Such kits can also, optionally, include one or more reagents for attaching the polynucleotides to the solid supports such as EDAC (see above).

The kits can optionally include, e.g., a control biological sample or control labeled-amplified nucleic acid containing known amounts of one or more microbial nucleic acids complementary to the detection oligonucleotides of the array.

In some embodiments, the kits can include one or more reagents for processing a biological sample. For example, a kit can include reagents for extracting RNA or DNA from a biological sample (e.g., glass beads and/or an extraction solution) and/or reagents for amplifying isolated RNA (e.g., reverse transcriptase, primers for reverse transcription (RT) or RT-polymerase chain reaction (PCR) amplification, or dNTPs) and/pr DNA. That is, the kits can include one or more primers containing, or consisting of, any of the polynucleotide sequences, or fragments thereof, depicted in Table 2.

TABLE 2

| Polynucleotide sequence | SEQ ID NO: |
|---|---|
| GACTCCTTGGTCCGTGTT | 34 |
| GAGTGAAAAAGTACGTGAAATTGTTGAAAGGGAA | 35 |
| CCCGCTGAACTTAAGCATATCAATAAGCGGAGGA | 36 |
| GACTCCTTGGTCCGTGTTTCAAGACG | 37 |

Additional primer sequences, which can be included in the kits described herein, are described in, e.g., PCT Publication No. WO 00/052203; Rijpkema et al. (1995) J. Clin. Microb. 33(12):3091-3095; and Anthony et al. (2000) J. Clin. Microb. 38:781-788, the disclosures of each of which are incorporated herein by reference in their entirety. One or more of the primers can be detectably labeled. For example, one or more primers can be detectably labeled, e.g., at the 5′ end, with any of the detectable-labels described herein such as digoxigenin.

The kits can also, optionally, contain one or more reagents for detectably-labeling RNA or DNA which reagents can include, e.g., an enzyme such as a Klenow fragment of DNA polymerase, T4 polynucleotide kinase, one or more detectably-labeled dNTPs, detectably-labeled gamma phosphate ATP (e.g., ³³P-ATP), or detectably-labeled (e.g., digoxigenin-labeled) primers (such as any of the primers described herein). The kits can include water (e.g., DNA or RNA-free water), one or more hybridizing solutions (e.g., SSC solutions; see below), and/or one or more sample vessels for storing or manipulating a biological sample, the extracted nucleic acid (e.g., extracted DNA or RNA), or amplicons of the extracted nucleic acid. Any of the reagents included in the kits can be DNA and/or RNA-free. Methods of rendering a composition DNA or RNA-free are known in art and include, e.g., UV-irradiating a composition for at least one (e.g., at least two, at least three, at least four, at least five, at least six, at least seven, or at least eight or more) hours.

Any of the kits described herein can also, optionally, include (or contain instructions on how to use) a flow-through membrane device such as the CodaXcel™ device, e.g., as described in U.S. Pat. Nos. 4,834,946; 4,713,349; 6,194,160; and 6,303,389, the disclosures of each of which are incorporated herein by reference in their entirety.

The kits described herein can also, optionally, include instructions for administering an appropriate anti-microbial treatment (e.g., an appropriate antibiotic, anti-fungal agent, anti-viral agent, or anti-protozoal agent) to a subject where the presence of one or more microorganisms in a biological sample (from the subject) has been detected using any of the arrays or kits described herein. For example, the kit can contain instructions for administering an anti-fungal agent (e.g., ketoconazole, fluconazole, or natamycin) when a biological sample from a subject has been determined to contain a fungus, or for administering any of a variety of antibiotics if the subject has been determined to have a bacteremia.

Samples and Sample Collection: As described above, a source (e.g., a first or second source) for use in the present methods can be virtually any source. For example, a source can be obtained from a biological setting, such as an organism. Sources can also be obtained from environmental, industrial, or other non-biological settings (e.g., non-living sources) and tested in the present methods. Thus, while a source can be derived from an organism (e.g., from a subject such as a human patient), the source can also be an artificial environment (e.g., a laboratory specimen, culture, or surface). In many cases, a source is a sample that contains or is suspected of containing one or more microorganisms (e.g., bacteria, fungus (e.g., yeast), protozoa, or virus) and, in most instances, those microorganisms will be unwanted in the tested source.

Moreover, the content of the source can be wholly or partially known or unknown. A source can contain, e.g., one or more known microorganisms or one or more microbial nucleic acids in a known or unknown quantity. For example, the source can contain one or more microorganisms (e.g., multiple different bacterial, fungal, and/or protozoal species) at a concentration of less than about 1,000 (e.g., less than about 900, less than about 800, less than about 700, less than about 600, less than about 500, less than about 400, less than about 300, less than about 250, less than about 200, less than about 150, less than about 100, less than about 50, less than about 25, less than about 20, less than about 15, less than about 10, less than about 9, less than about 8, less than about 7; less than about 6, less than about 5, less than about 4, less than about 3 or less) colony forming units (cfu) per milliliter (ml). Where, e.g., the source contains one or more viral microorganisms, the source can contain at least one virus at a concentration of less than about 1,000 (e.g., less than about 900, less than about 800, less than about 700, less than about 600, less than about 500, less than about 400, less than about 300, less than about 250, less than about 200, less than about 150, less than about 100, less than about 50, less than about 25, less than about 20, less than about 15, less than about 10, less than about 9, less than about 8, less than about 7, less than about 6, less than about 5, less than about 4, less than about 3 or less) plaque forming units (pfu) per ml. A source can contain any type of microorganism (e.g., any of the microorganisms described herein).

A sample can contain two or more (e.g., two, three, four, five, six, seven, eight, nine, 10, 11, 12, 13, 14, 15, 17, 18, 19, 20, 21, 22, 23, 24, 25, or 30 or more) different microorganisms. For example, a sample can contain one or more bacteria and fungi, bacteria and virus, bacteria and protozoa, fungi and virus, or any combination thereof. A sample can contain two or more different species of the same genus of microorganism. For example, a sample can contain two different Candida species (e.g., Candida albicans and Candida glabrata) and/or two different species of Staphylococcus (e.g., Staphylococcus haemolyticus and Staphylococcus aureus). A sample can also contain two or more different strains of the same microbial species, e.g., two different E. coli strains.

A sample can be a food sample, a water sample, or an air sample. For example, a sample can be, or be obtained from, a food product or water product suspected of being contaminated by a microorganism (e.g., a well water sample or a sample of vegetable produce (e.g., spinach or scallions) suspected of being contaminated with a bacterium such as E. coli or a virus such as hepatitis). In the case of airborne contamination, or the suspicion of airborne contamination, a sample can be a sample collected in an air vent or room of a building so suspected of being contaminated with a microorganism such as Legionella pneumophila or Streptococcus pneumoniae.

A sample can be a biological sample. Suitable biological samples for the methods described herein include any biological fluid, cell, tissue, or fraction thereof, which includes one or more microorganisms or biomolecules (e.g., microbial DNA or RNA) of interest. A biological sample can be, for example, a specimen obtained from a subject (e.g., a bird, an insect, a reptile, a fish, or mammal (e.g., a rat, mouse, gerbil, hamster, cat, dog, goat, pig, cow, bat, horse, non-human primate, or a human)) or can be derived from such a subject. For example, a biological sample can be a tissue section obtained by biopsy, or cells that are placed in or adapted to tissue culture. A biological sample can also be, or include, a biological fluid such as urine, blood, plasma, serum, stool, saliva, milk, sweat, semen, cerebral spinal fluid, tears, wound exudates, skin scrapings, or mucus, or such a sample absorbed onto a paper or polymer substrate. A biological sample can be, or include, a pulmonary sample such as, e.g., a sputum sample, a broncheolar lavage sample, an endotracheal aspirate sample, an upper-respiratory mucosal swab, or a protected specimen brush sample. A biological sample can be further fractionated, if desired, to a fraction containing particular cell types. For example, a blood sample can be fractionated into serum or into fractions containing particular types of blood cells such as red blood cells or white blood cells (leukocytes). The sample can also be of plasma or a synthetic or partially synthetic blood product. In some embodiments, two or more samples (e.g., the first and second sample) can be different types of samples from the same sample or subject. For example, two or more different biological samples such as blood, mucous, sputum, urine, stool, sweat, cerebral-spinal fluid, tears, and/or semen can be obtained from the same subject and analyzed using the methods described herein.

In some embodiments, the methods can include the step of obtaining a biological sample from a subject (e.g., a mammal such as a human) or other source. The subject can have, be suspected of having, or be at risk of developing, an infection by any microorganism described herein. Methods for obtaining a biological sample include, e.g., phlebotomy, swab (e.g., buccal swab or drag swab), fine needle aspirate biopsy procedure, broncheolar lavage, endotracheal aspirate, or a protected specimen brush. Biological samples can also be collected, e.g., by microdissection (e.g., laser capture microdissection (LCM) or laser microdissection (LMD)), bladder wash, smear (PAP smear), urine collection, or ductal lavage.

Methods for obtaining and/or storing samples that preserve the integrity of, e.g., nucleic acids in the sample are well known to those skilled in the art. For example, a biological sample can be further contacted with one or more additional agents such as appropriate buffers and/or inhibitors, including nuclease, protease, or phosphatase inhibitors, which preserve or minimize changes in the molecules (e.g., nucleic acids or proteins) in the sample. Such inhibitors include, for example, chelators such as ethylenediamne tetraacetic acid (EDTA), ethylene glycol bis(P-aminoethyl ether) N,N,N1,N1-tetraacetic acid (EGTA). Appropriate buffers and conditions for isolating molecules are well known to those skilled in the art and can be varied depending, for example, on the type of molecule in the sample to be characterized (see, for example, Ausubel et al. Current Protocols in Molecular Biology (Supplement 47), John Wiley & Sons, New York (1999); Harlow and Lane, Antibodies: A Laboratory Manual (Cold Spring Harbor Laboratory Press (1988); Harlow and Lane, Using Antibodies: A Laboratory Manual, Cold Spring Harbor Press (1999); Tietz Textbook of Clinical Chemistry, 3rd ed. Burtis and Ashwood, eds. W.B. Saunders, Philadelphia, (1999)). A sample also can be processed to eliminate or minimize the presence of interfering substances. For example, a biological sample can be fractionated or purified to remove one or more materials that are not of interest. Methods of fractionating or purifying a biological sample include, but are not limited to, chromatographic methods such as liquid chromatography, ion-exchange chromatography, size-exclusion chromatography, or affinity chromatography.

For use in the methods described herein, a sample can be in a variety of physical states. For example, a sample can be a liquid or solid, can be dissolved or suspended in a liquid, can be in an emulsion or gel, and can be absorbed onto a material.

Exemplary biological samples, methods for obtaining the samples, and purification methods (e.g., nucleic acid extraction) are detailed in the accompanying Examples.

A sample can be processed to facilitate extraction of nucleic acids. For example, if the sample includes cells or other biological structures, the sample can be treated with freeze/thaw treatment, drying and rehydrating, a dounce, lysis buffer (e.g., one with a detergent), glass beads, or other methods (see the accompanying Examples).

Applications: The methods and compositions (e.g., arrays and kits) described herein can be used to, e.g., (a) detect the presence or absence of one or more microorganisms in a sample (e.g., a biological sample from a subject); (b) determine the identity of one or more microorganisms in a sample; and/or determine (e.g., select and/or administer) the appropriate therapeutic modality for a subject so determined to be infected with one or more microorganisms.

Methods for Identifying a Microorganism in a Source: The invention features methods for identifying one or more microorganisms in at least two source samples. The methods can optionally include the step of extracting nucleic acid from a source. Methods for extracting nucleic acid (e.g., the first or second nucleic acid) from a source vary, in part, on the nature of the source and the nucleic acid (e.g., microbial DNA or microbial RNA) being extracted. For example, DNA can be extracted from a source, e.g., a biological sample, by contacting the source with a lysis buffer including one or more detergents (e.g., saponin, sodium dodecyl sulfate, deoxycholine, NP-40, Tween-20, or Triton X-100). In some instances, the extraction can also involve mechanical disruption. For example, a mixture of particles (e.g., glass beads) can be mixed with the source along with the lysis buffer to aid in disrupting cell membranes (e.g., by vortexing or other mechanical forces). The lysis buffer can also include one or more proteases (e.g., proteinase K) and an RNAase. Following lysis, the extraction process can include precipitating the isolated DNA using, e.g., cold alcohol (e.g., ethanol), a salt (e.g., sodium or potassium acetate), and optionally a carrier such as glycogen. After precipitating the DNA, the DNA can be washed with alcohol and then resuspended in an appropriate storage buffer (e.g., Tris-EDTA (TE), pH. 8.0).

Methods for extracting RNA from a source (e.g., a biological sample) are similar to those described above for DNA and can include contacting the source with a lysis buffer including one or more detergents, RNase-free DNase, and RNase-free proteases (as above). The extraction can also include mechanical disruption techniques. Following the lysis, the RNA can be isolated by precipitating the isolated RNA, washing the precipitated RNA, and resuspending the RNA in an appropriate storage buffer, which generally contains one or more RNase inhibitors (such as diethylpyrocarbonate (DEPC)) and can be maintained at a neutral or non-basic pH.

Suitable methods for extracting nucleic acid from a source are further described in, e.g., Sambrook et al. Molecular Cloning: A Laboratory Manual Second Edition vol. 1, 2 and 3. Cold Spring Harbor Laboratory Press: Cold Spring Harbor, N.Y., USA, November 1989; the disclosure of which is incorporated herein by reference in its entirety.

In some embodiments, none of the sources (e.g., the first nor the second source) is subjected to any process that would promote propagation of the microorganism. That is, prior to extraction, a source is not subjected to any process that would promote propagation or expansion (through microbial cell division or viral reproduction) of one or more microorganisms suspected of being present in the source. Examples of such a process include, e.g., culturing of the source and/or in embodiments where the source contains (or is suspected of containing) a virus, contacting a population of cultured cells (e.g., host cells) with the source. In some embodiments, nucleic acid is extracted from a source within at least about 24 (e.g., at least about 23, at least about 22, at least about 21, at least about 20, at least about 19, at least about 18, at least about 17, at least about 16, at least about 15, at least about 14, at least about 13, at least about 12, at least about 11, at least about 10, at least about 9, at least about 8, at least about 7, at least about 6, at least about 5, at least about 4, at least about 3, at least about 2, at least about 1, or less than 1) hours after obtaining the source. Nucleic acid can be extracted from a source less than 60 (e.g., less than 55, less than 50, less than 45, less than 40, less than 35, less than 30, or less than 20 or less) minutes after obtaining the source. Samples may also be stored (e.g., frozen) for later analysis or re-testing.

In embodiments where the sources contain (or are suspected of containing) one or more fungi, the nucleic acid can be extracted from a source without first enriching any of the fungi in the source. For example, the source is not subjected to any conditions which would further isolate one or more fungi, if present, in the source.

In some embodiments, two or more samples, nucleic acid samples, or amplified nucleic acid samples are not combined (“pooled”) prior to contacting the samples with an array.

Following the extraction, a nucleic acid sample can be stored under conditions that do not promote propagation or expansion of a microorganism, e.g., the sample can be frozen.

Suitable methods for amplifying at least one selected region of sequence in the nucleic acid are known in the art and set forth in the accompanying Examples. Suitable selected regions of sequence for amplification include, but are not limited to, ribosomal RNA (rRNA) or DNA encoding rRNA such as bacterial small subunit (e.g., 16S) or large subunit (e.g., 23S) rDNA or fungal small subunit (e.g., 18S) or large subunit (e.g., 26S) rDNA. DNA extracted from a source can be amplified in a variety of ways including a standard DNA polymerase reaction or a polymerase chain reaction (PCR). Extracted RNA from a biological sample can be amplified, e.g., using reverse-transcriptase polymerase chain reaction (RT-PCR). Primers suitable for amplifying extracted nucleic acid are set forth in the Examples and are also depicted in Table 2 (above). For example, extracted bacterial 23S rDNA can be amplified using the following forward and reverse primer set: forward: 5′GCGATTTCYGAAYGGGGGRAACCC3′ (SEQ ID NO:38) and reverse: 5′TTCGCCTTTCCCTCACGGTAT3′ (SEQ ID NO:39). Extracted fungal (e.g., yeast) 26S rDNA can be amplified using the following forward and reverse primers sets: U2 (forward): GACTCCTTGGTCCGTGT™ (SEQ ID NO:34) and U1c (reverse): GAGTGAAAAAGTACGTGAAATTGTTGAAAGGGAA (SEQ ID NO:35) or D1long1: CCCGCTGAACTTAAGCATATCAATAAGCGGAGGA (SEQ ID NO:36) and D2Rlong1 (reverse): GACTCCTTGGTCCGTGTTTCAAGACG (SEQ ID NO:37).

As will be clear from the foregoing, the primer sets used to amplify extracted nucleic acid can be capable of binding to nucleic acid sequences that are highly conserved throughout a phylum, class, order, family, genus, and/or species of microorganism. For example, a “broad-range” primer set can be used to amplify a wide range of diverse bacteria such as Staphylococcus, Streptococcus, Enterococcus, and Echererichia (e.g., using the primers above having SEQ ID NOs: 38 or 39). In some embodiments, a primer set can be used to amplify a subset of related organisms (e.g., a primer set that binds to sequences conserved in a genus of bacteria such as Staphylococcus). In some embodiments, a primer set can be used that binds to nucleic acid sequences in specific microbes (e.g., E. coli) or a range of strains of a particular microbe (e.g., two or more strains of E. coli). It is understood that multiple primer sets (e.g., bacteria-specific and fungus-specific primer sets) can be used amplify extracted nucleic acids from different groups of microorganisms in the same reaction.

The step of amplifying (if a selected region of nucleic acid sequence is present) at least one selected region of nucleic acid sequence in each of the first and second nucleic acid samples can be performed under conditions that permit detection of the amplified first or second nucleic acid sequence if the concentration of the microorganism exceeds a threshold concentration. The conditions can include varying, e.g., the number of PCR cycles used to amplify the selected region(s), the amount of nucleic acid sample used for amplification, the temperature at which the amplification is performed, or the concentration of primers added to the amplification reaction).

In some embodiments, the step of contacting the array can be performed under conditions that permit detection of the amplified first or second nucleic acid sequence if the concentration of the microorganism exceeds a threshold concentration. The conditions can include, e.g., varying: (i) the amount of amplified first or second nucleic acid contacted with the array; (ii) the temperature at which the first or second nucleic acid is contacted with the array; or (iii) the concentration or binding efficiency of the detection oligonucleotides on the porous solid support.

The threshold concentration can be, e.g., at least or about 10², 10³, 10⁴, or between about 10⁵-10⁶ cfu/mL. The threshold can depend on, e.g., the type of source. For example, the threshold can be about 10³ cfulmL for a protected specimen brush sample, about 10⁴ cfu/mL for a bronchoalveolar lavage sample, or between about 10⁵-10⁶ cfu/mL cfu/mL for a endotracheal aspirate sample.

In some embodiments, the amplified first or second nucleic acids can be detectably labeled. The nucleic acids can be detectably labeled during amplification or following amplification. For example, an PCR or reverse transcription-PCR (RT-PCR) amplification step can be used to detectably-label the amplified nucleic acid, e.g., using detectably labeled primers. Alternatively, the amplified nucleic acids can be labeled during amplification by using detectably labeled nucleotides (e.g., nucleotide analogues or radiolabeled nucleotides). A detectable label can be enzymatically (e.g., by nick-translation or kinase (e.g., T4 polynucleotide kinase)) or chemically conjugated to the amplified nucleic acid following amplification (see, e.g., Sambrook et al. (supra)). Detectable labels include, e.g., fluorescent labels (e.g., umbelliferone, fluorescein, fluorescein isothiocyanate, rhodamine, dichlorotriazinylamine fluorescein, dansyl chloride, allophycocyanin (APC), or phycoerythrin); luminescent labels (e.g., europium, terbium, or Qdot™ nanoparticles); radionuclide labels (e.g., ¹²⁵I, ¹³¹I, ³⁵S, ³²P, ³³P, or ³H); chemical labels (see, e.g., U.S. Pat. Nos. 4,582,789 and 4,563,417); a label that recruits an enzyme; or labels detectable by an antibody or ligand-binding proteins specific for the detectable label (e.g., digoxigenin and biotin).

To identify a microorganism in the source, the amplified nucleic acids (e.g., detectably-labeled amplified nucleic acids) can be contacted to an array of detection oligonucleotides (e.g., any nucleic acid array described herein). Hybridization of the amplified nucleic acid to detection oligonucleotide complementary to the amplified nucleic acid indicates identifies the one or more microorganisms present in the source.

Depending on the specific application, varying hybridization conditions can be employed to achieve varying degrees of selectivity of a detection oligonucleotide towards target sequence. Standard stringency conditions are described by Sambrook, et al. (supra) and Haymes, et al. Nucleic Acid Hybridization, A Practical Approach, IRL Press, Washington, D.C. (1985), the disclosures of both of which are incorporated herein by reference in their entirety. In order for a nucleic acid molecule to serve as a primer or detection oligonucleotide, it need only be sufficiently complementary in sequence to be able to form a stable double-stranded structure under the particular hybridization conditions (e.g., solvent and salt concentrations) employed.

Appropriate stringency conditions that promote DNA hybridization, for example, 5.0× sodium chloride/sodium citrate (SSC) at about 50° C. for about 45 minutes to 1 hour, followed by a wash of 0.25-2×SSC at 50° C., are known to those skilled in the art or can be found in, e.g., PCT Publication No. WO 00/052203; Paster et al. (1998) Methods in Cell Science 20:223-231; Anthony et al. (2000) J. Clin. Microb. 38:781-788; Ausubel, et al., Current Protocols in Molecular Biology, John Wiley & Sons, N.Y. (1989), the disclosure of which is incorporated herein by reference in its entirety. For example, the salt concentration in the wash step can be selected from a low stringency of about 2.0×SSC at 50° C. to a high stringency of about 0.2×SSC at 50° C. The temperature used in the wash step can be increased from low stringency conditions at room temperature (about 22° C.) to high stringency conditions at about 65° C. Temperature and salt conditions may be varied independently.

Methods of detecting and/or for quantifying a detectable label depend on the nature of the label and are known in the art. Examples of detectors suitable for detecting such detectable labels include, without limitation, x-ray film, radioactivity counters, scintillation counters, spectrophotometers, colorimeters, fluorometers, luminometers, and densitometers.

In embodiments where the detectable-label is one that is recognized by an antibody specific for the label, detection of the detectable label generally involves use of the antibody. In one example, an immunoassay can be used for detecting the binding of a labeled amplified nucleic acid to an array. The immunoassay can be performed with a primary antibody specific for the detectable label and that bears a detection moiety (e.g., a fluorescent agent or enzyme). Alternatively, the primary antibody can be unlabeled and a detectably-labeled secondary antibody that specifically binds the primary antibody can be used to detect the binding of nucleic acid to the array. The presence or amount of bound detectably-labeled antibody indicates the presence or amount of the nucleic acid (and the corresponding microorganism) in the sample.

Methods for generating antibodies or antibody fragments specific for a antigen encoded include immunization, e.g., using an animal, or by in vitro methods such as phage display.

An antigen can be used to prepare antibodies by immunizing a suitable subject, (e.g., rabbit, goat, mouse, or other mammal) with the peptide. An appropriate immunogenic preparation can contain, for example, a chemically synthesized antigen or a recombinantly expressed antigen (e.g., where the antigen is a nucleic acid of a peptide). The preparation can further include an adjuvant, such as Freund's complete or incomplete adjuvant, or similar immunostimulatory agent. Immunization of a suitable subject with an immunogen preparation induces a polyclonal anti-peptide antibody response.

The term antibody as used herein refers to immunoglobulin molecules and immunologically active portions of immunoglobulin molecules (i.e., molecules that contain an antigen binding site that specifically bind to the antigen). An antibody that specifically binds to an antigen described herein is an antibody that binds the antigen, but does not substantially bind other molecules in a sample. Examples of immunologically active portions of immunoglobulin molecules include F(ab) and F(ab′)₂ fragments.

The antibody can be a monoclonal antibody or a preparation of polyclonal antibodies. The term monoclonal antibody, as used herein, refers to a population of antibody molecules that contain only one species of an antigen binding site capable of immunoreacting with the antigen. A monoclonal antibody composition thus typically displays a single binding affinity for a particular antigen with which it immunoreacts.

Polyclonal antibodies can be prepared as described above by immunizing a suitable subject with an immunogen. The antibody titer in the immunized subject can be monitored over time by standard techniques, such as with an enzyme linked immunosorbent assay (ELISA) using immobilized peptide. If desired, the antibody molecules directed against the antigen can be isolated from the mammal (e.g., from the blood) and further purified by techniques such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), or the EBV-hybridoma technique (Cole et al. (1985), Monoclonal Antibodies and Cancer Therapy, Alan R. Liss, Inc., pp. 77-96). Any of the many well known protocols used for fusing lymphocytes and immortalized cell lines can be applied for the purpose of generating an monoclonal antibody (see, e.g., Current Protocols in Immunology, supra; Galfre et al. (1977) Nature 266:55052; R. H. Kenneth, in Monoclonal Antibodies: A New Dimension In Biological Analyses, Plenum Publishing Corp., New York, N.Y. (1980); and Lerner (1981) Yale J. Biol. Med., 54:387-402).

As an alternative to preparing monoclonal antibody-secreting hybridomas, a monoclonal antibody can be identified and isolated by screening a recombinant combinatorial immunoglobulin library (e.g., an antibody phage display library) with a peptide described herein to isolate immunoglobulin library members that bind the peptide.

An antibody can itself be optionally coupled to a detectable label (e.g., colorimetric detection label) such as an enzyme (e.g., alkaline phosphatase, horseradish peroxidase, luciferase/luciferin, or any of those described herein. The antibody can be coupled to a first or second member of a binding pair (e.g., streptavidin/biotin or avidin/biotin), the second member of which can be conjugated to a detectable label.

Methods for detecting one or more nucleic acids in a sample can be performed in formats that allow for rapid sample preparation, processing, and, as discussed above, analysis of multiple samples in parallel. This can be, for example, using a flow-through device (as described herein). Stock solutions for various reagents can be provided manually or robotically, and subsequent sample preparation (e.g., PCR or RT-PCR, labeling, or sample extraction), pipetting, diluting, mixing, distribution, washing, incubating (e.g., hybridization), sample readout, data collection (optical data) and/or analysis (computer aided image analysis) can be done robotically using commercially available analysis software, robotics, and detection instrumentation capable of detecting the signal generated from the assay. Examples of such detectors include, but are not limited to, spectrophotometers, luminometers, fluorimeters, and devices that measure radioisotope decay.

Flow-through devices for use in any of the present methods are described herein. One exemplary device for use in any of the methods or with any of the compositions described herein is the CodaXcel™ (Immunetics, Boston, Mass.), which is described, for example, in U.S. Pat. Nos. 6,194,160 and 6,303,389, the disclosures of each of which are incorporated by reference in their entirety. The various wash, hybridization, and detection steps (e.g., those using antibodies and colorimetric indicators) of the methods can be performed using such devices by passing the solutions through the membrane with the aid of negative pressure applied to the membrane.

Additional methods of detecting amplified include, e.g., northern blot or southern blot techniques, which are described in detail in Sambrook et al. (supra).

The methods (and the compositions) can be used to determine varying levels of information from a sample. For example, the methods and compositions can be used to discriminate between different types of microorganisms such as fungus, bacteria, viruses, or protozoa. The methods and compositions can also be used to sub-group a particular type of microorganism, e.g., distinguishing between a Gram negative or Gram positive bacteria, or can be used to determine the identity of a particular species of microorganism, e.g., Candida albicans versus Candida glabrata. The methods and compositions described herein allow for additional discrimination, e.g., between strains of a given species of microorganism (e.g., different strains of E. coli). Such discrimination can allow for, e.g., identifying the presence of drug resistant forms of a microorganism in a sample (e.g., antibiotic or multi-drug resistant Tuberculosis, Staphylococcus, or E. coli or multi-drug resistant HIV).

It is understood that such methods can be useful in a variety of applications including, e.g.: (a) statistical analyses of infections in patient or general population cohorts, (b) determining the rate or prevalence of a drug resistant microorganism in a population over time, (c) identifying causative agents underlying nosocomial infections (e.g., hospital acquired pneumonia), (c) detecting genetic variation of a microbe in a population of hosts, or (d) selecting an appropriate therapeutic modality for a subject based on the specific microorganism so identified in a biological sample from the subject (see “Selecting an Anti-microbial Treatment Regimen”).

The methods can be used to identify one or more (e.g., one, two, three, four, five, six, seven, eight, nine, 10, 11, 12, 13, 14, 15, 16, 17, 18, 19, or 20 or more) different microorganisms in each of one, or two or more (e.g., two, three, four, five, six, seven, eight, nine, 10, 11, 12, 13, 14, 15, 20, 25, or 30 or more) different samples. The method can be configured to identify the one or more different microorganisms in a single sample or the method can be configured to identify a single type of microorganism in each of two or more (e.g., two, three, four, five, six, seven, eight, nine, 10, 11, 12, 13, 14, 15, 20, 25, or 30 or more) different sources.

Samples can be tested singly, but an advantage of the present methods is the ability to assess multiple samples essentially simultaneously or in parallel. Further, the selection of detection oligonucleotides can be varied to allow one to assess the nucleic acid content in several different samples obtained from the same subject (e.g., samples obtained at different times (e.g., over the course of treatment) or from different locations (e.g., a blood sample and a sample obtained by bronchial/alveolar lavage)). For example, nucleic acid can be extracted from a single sample of blood from a single subject at a single time and contacted to a plurality of detection oligonucleotides to simultaneously identify one or more microorganisms (one or more microbial nucleic acids), the genotype of one or more particular microorganisms, and/or the presence or absence of one or more antibiotic resistance or virulence genes within one or more of the microorganisms. For example, the method can be configured such that the identity of Klebsiella pneumonia can be determined as well as whether or not the bacterium contains a gene encoding a KPC-1 or KPC-2 carbapenemase.

As noted, multiple samples from the same patient or different patients may be tested at the same time. The two or more samples can be, e.g., from two or more different sources or subjects. This can be useful, e.g., in screening a large cohort of subjects for one or more microorganisms (e.g., a group of individuals exposed to, or suspected of being exposed to, a microbial pathogen such as HIV or anthrax). Alternatively, the two or more sources can be different types of samples from the same source or subject. For example, biological samples of blood, mucous, sputum, bronchoalveolar lavage, urine, stool, sweat, cerebral-spinal fluid, tears, and/or semen (or any other biological sample described herein) can be obtained from the same subject and analyzed using the methods and compositions described herein. Detecting multiple samples in parallel can be performed using the CodaXcel device (described above) and checkerboard detection techniques (see, e.g., Paster et al. (1998) Methods in Cell Science 20:223-231, the disclosure of which is incorporated herein by reference in its entirety).

The methods and compositions can also be used to, e.g., simultaneously determine several different parameters from a single sample. For example, nucleic acid can be extracted from a single blood sample from a single subject and contacted to a plurality of detection oligonucleotide sets to simultaneously detect the presence or absence of one or more microorganisms (one or more microbial nucleic acids), the genotype of one or more particular microbes, and/or the presence or absence of an antibiotic resistance gene within one or more of the microbes. An exemplary methods of simultaneously determining one or more parameters from a sample (e.g., a biological sample) is a checkerboard technique (see above). For example, a polynucleotide array can be designed that includes two or more distinct groups of detection oligonucleotide sets (e.g., each column or row contains a different group of detection oligonucleotide sets, or one or more quadrants of a polynucleotide array contains a different group of detection oligonucleotide sets). The distinct detection oligonucleotide sets can, e.g., contain one or more oligonucleotides specific for different microbes (e.g., different fungi, different bacteria, different protozoa, or different viruses), different species of a given type of microbe, different strains of a specific species of microbe, an antibiotic resistance marker present within a microbe, or any combination of the foregoing. The extracted nucleic acid (or amplicons thereof) can be simultaneously contacted to each distinct oligonucleotide set in parallel, thereby allowing simultaneous determination of multiple parameters.

In some embodiments, the methods can be sensitive enough to detect the presence of a microbial nucleic acid at a concentration of less than 30 molecules (e.g., less than 29 molecules, less than 28 molecules, less than 27 molecules, less than 26 molecules, less than 25 molecules, less than 24 molecules, less than 23 molecules, less than 22 molecules, less than 21 molecules, less than 20 molecules, less than 19 molecules, less than 18 molecules, less than 17 molecules, less than 16 molecules, less than 15 molecules, less than 14 molecules, less than 13 molecules, less than 12 molecules, less than 11 molecules, less than 10 molecules, less than nine molecules, less than eight molecules, less than seven molecules, less than six molecules, less than five molecules, less than four molecules, less than three molecules, less than two molecules, or less than one molecule) per ml of sample. In some embodiments, the methods can be sensitive enough to detect the presence of a microbial nucleic acid at a concentration of one or two molecules per ml of sample. In some embodiments, the methods described herein can be sensitive enough to detect the presence of one or more microorganisms (e.g., multiple different bacterial or fungal species) at a concentration of less than about 1,000 (e.g., less than about 900, less than about 800, less than about 700, less than about 600, less than about 500, less than about 400, less than about 300, less than about 250, less than about 200, less than about 150, less than about 100, less than about 50, less than about 25, less than about 20, less than about 15, less than about 10, less than about 9, less than about 8, less than about 7, less than about 6, less than about 5, less than about 4, less than about 3 or less) colony forming units (cfu) per milliliter (ml). Where, e.g., the composition contains one or more viral microorganisms, the methods can be sensitive enough to detect the presence of at least one virus at a concentration of less than about 1,000 (e.g., less than about 900, less than about 800, less than about 700, less than about 600, less than about 500, less than about 400, less than about 300, less than about 250, less than about 200, less than about 150, less than about 100, less than about 50, less than about 25, less than about 20, less than about 15, less than about 10, less than about 9, less than about 8, less than about 7, less than about 6, less than about 5, less than about 4, less than about 3 or less) plaque forming units (pfu) per ml.

Use of Methods in Conjunction with a Flow-Through Device: Any of the methods described herein can be performed in conjunction with a flow-through device. As used herein, the terms “flow-through device” and “membrane flow-through device” are used interchangeably. A flow-through device can comprise a number of parts including a cartridge, or cassette, and a plate for receiving the cassette. The cassette is generally configured such that it can house a porous solid support (e.g., a membrane such as a nitrocellulose membrane or a nylon membrane). The device can also provides a means for producing negative pressure applied to the porous solid support such that liquids contacted to the surface of the support are actively pulled through the support. The source of negative pressure can be, e.g., a vacuum apparatus operably-linked to, or directly built into, the device.

The amount of time in which a liquid remains on the surface of the support can vary depending on the amount of negative pressure applied. In some embodiments, the device can be configured to include a operator means for adjusting the negative pressure applied to the support. The means can be in the form of a control unit. For example, the device can include a control unit containing a switch for alternating between, e.g., a “fast” or “slow” pace at which a liquid is pulled through the support. In another example, a control unit of the device can include a rheostat such that a fine-tuned control of the speed at which a liquid traverses the support can be maintained. In some embodiments, the control unit is, or comprises, a computer and can be programmed to adjust negative pressure according to a predetermined schedule. In some embodiments, the computer can contain a programmable memory such that programs can be stored and called back by an operator.

In some embodiments, the device features a means for agitating the cassette. Such a means can be useful for mixing a solution of multiple reagents applied to the support or for increasing the potential for an interaction between a component of the solution and the support (or a component of the support such as an oligonucleotide attached to the support). The means can be implemented by way of a control unit containing one or more of a switch, a rheostat, and/or a computer as described above. Suitable configurations of an agitator for use in the device, e.g., ultrasonic transmitters, magnetic devices, or any other element suitable for moving the plate containing the cassette can be found in, e.g., U.S. Pat. No. 6,194,160.

In some embodiments, the action of both the agitator and the negative pressure facilitate the passage of a liquid through the support.

In some embodiments, the device can feature a means for controlling the amount of negative pressure applied to the support. The means can control the pressure in a number of ways including, but not limited to, controlling the power to a pump proving the vacuum or controlling an aperture of a hose or tubing connecting a pump to the device. The means can be implemented by way of a control unit containing one or more of a switch, a rheostat, and/or a computer as described above.

In some embodiments, the device can include a waste container coupled in fluid communication with the source of negative pressure (e.g., a pump) capable of receiving waste fluid diffused through the support. The waste fluid can be, e.g., a wash buffer, a hybridization buffer, or a detection buffer. The waste fluid can be, e.g., biohazardous or radioactive.

In some embodiments, the cassette can configured such that all or substantially all of the surface of a porous solid support is accessible to an operator. In some embodiments, the cassette can include an array of two or more (e.g., two, three, four, five, six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 or more) channels in which an operator is provided access to the support. Such channels can be useful in an assay in which more than one sample is contacted to a single support in parallel. The channels offer a physical barrier that can prevent the mixing of two or more samples on the support. In some embodiments, the cassette includes an array of 96 or 384 channels. The channels can be of a variety of shapes and sizes. For example, the channels can be linear or circular. Two or more of the channels can be uniform in size and/or shape or can be of different sizes and/or shapes. In some embodiments, each channel of the array is of the same size and shape. The channels can be of varying depth. In some embodiments, each of the channels of the array are the same depth.

In the context of the methods described herein, the number of channels can be directly proportional to the number of samples to be contacted to a single porous solid support.

In some embodiments, the cassette can include a base and an upper plate, wherein the porous solid support is placed between the base and upper plate and the upper plate contains two or more channels. A cassette can be configured such that many different upper plates can be used in connection with a common base. For example, a base can be compatible with a first upper plate containing an array of 96 channels and a second upper plate containing an array of 384 channels. The upper plate and the base can be held together by a number of means. For example, the base and upper plate can be held together using one or more screws. The upper plate and base can be held together through one or more interlocking pairs such as a male and female adapter. In some embodiments, the upper plate and base are held together by means of the negative pressure applied to the membrane.

Exemplary flow-through devices, cassettes, and are described in, e.g., U.S. Pat. Nos. 6,194,160; 6,303,389; 5,100,626; 4,978,507; 4,834,946; and 4,713,349, the disclosures of each of which are incorporated herein by reference.

Selecting an Anti-Microbial Therapeutic Regimen: Following the identification of one or more microorganisms (e.g., one or more pathogenic microorganisms in a biological sample from a subject), a medical practitioner (e.g., a doctor) can select the appropriate anti-microbial treatment regimen for the subject (e.g., an antibiotic, an anti-fungal, an anti-viral, or anti-protozoal agent). Selecting a therapy for a subject can be, e.g.: (i) writing a prescription for a medicament; (ii) giving (but not necessarily administering) a medicament to a subject (e.g., handing a sample of a prescription medication to a patient while the patient is at the physician's office); (iii) communication (verbal, written (other than a prescription), or electronic (e.g., email or post to a secure site)) to the patient of the suggested or recommended anti-microbial treatment regimen (e.g., an antibiotic); or (iv) identifying a suitable anti-microbial treatment regimen for a subject and disseminating the information to other medical personnel, e.g., by way of patient record. The latter (iv) can be useful in a case where, e.g., more than one therapeutic agent are to be administered to a patient by different medical practitioners.

After selecting an appropriate anti-microbial therapeutic regimen for an infected subject, a medical practitioner can administer the appropriate anti-microbial therapeutic regimen (e.g., a regimen comprising one or more anti-microbial agents) to the subject. In other embodiments, the anti-microbial therapeutic regimen can be administered by a subject that is not a medical practitioner (e.g., the anti-microbial treatment regimen can be self administered). Suitable anti-microbial therapeutic agents (e.g., antibacterial agents, anti-fungal agents, or anti-viral agents) include, e.g., aminoglycosides (e.g., amikacin, gentamicin, kanamycin, neomycin, netilmicin, streptomycin, or tobramycin); ansamycins, cephalosporins (e.g., cefaclor, cefamandole, cefoxitin, or cefprozil); macrolides (e.g., azithromycin, clarithromycin, erthyromycin, or roxithromycin); penicillins (e.g., amoxicillin, ampicillin, azlocillin, carbenicillin, penicillin, piperacillin, or ticarcillin); quinalones (e.g., ciprofloxacin, enoxacin, levofloxacin, ofloxacin, or moxifloxacin); tetracyclines (e.g., doxycycline, micocycline, or tetracycline); imidazoles (e.g., miconazole, ketoconazole, clotrimazole, econazole, bifonazole, butoconazole, or fenticonazole); triazoles (e.g., fluconazole, itraconazole, isavuconazole, ravuconazole, posaconazole, voriconazole, or terconazole); anti-virals (e.g., abacavir, acyclovir, acyclovir, adefovir, amantadine, amprenavir, arbidol, atazanavir, atripla, ganciclovir, gardasil, indinavir, inosine, Integrase inhibitor, interferon, nucleoside analogues, penciclovir, protease inhibitors, reverse transcriptase inhibitors, or saquinavir); and anti-protozoals including nitazoxanide, metronidazole, eflornithine, furazolidone, hydroxychloroquine, iodoquinol, and pentamidine.

Methods of administering anti-microbial agents are known in the medical arts. Such agents can be administered in conjunction with one or more additional therapies for treating an infection. For example, a subject with sepsis can be administered the selected one or more antibiotics in conjunction with any one or more of hemodialysis, mechanical ventilation, transfusion, surgical drainage, fluid replacement, oxygen, or an anti-inflammatory (such as a TNF alpha inhibitor). Any of these above-described therapeutic modalities can include one or more treatments for side-effects of a anti-microbial agent including, e.g., an anti-nausea medication.

### EXAMPLES

### Example 1

### Extraction of DNA from Whole Blood

The following materials were used in the studies described below: Qiagen QiaAmp mini DNA minikit (Qiagen Ref: 51304; Qiagen, Valencia, Calif.); Qiagen ATL buffer (tissue lysis buffer; Qiagen Ref: 19076); 0.5 μm glass beads (Sigma Ref: G8772; Sigma, St. Louis, Mo.); screw-cap 2 ml sample tubes; phosphate-buffered saline (PBS; Accugene VWR 12001-764); proteinase K, (Roche Ref: 3115828001; Roche, Indianapolis, Ind.); 10% saponin (Sigma Ref: S4521); absolute 200 proof ethanol; and 1.5 ml Eppendorf biopure tubes (Eppendorf; Westbury, N.Y.).

First, 1.8 to 2 ml of a whole blood sample was transferred to a 2 ml screwcap tube. Twenty microliters (μl) of 10% saponin was added to the sample and subsequently mixed by vortexing. The sample was incubated for 1 minute at room temp, vortexed again, and then subjected to centrifugation at 14,000 rpm for 5 minutes. The supernatant was removed from the pellet. The pellet was washed with 1 ml of PBS (containing 10 μl of saponin) and then subjected to centrifugation at 14,000 rpm for 5 minutes. The supernatant was removed and the pellet was washed (with 1 ml of PBS) and centrifuged (14,000 rpm) a second time. Following removal of the supernatant, 350 μl of ATL buffer (see above) was added to the pellet along with 0.5 ml of glass beads. The sample was subjected to bead beating (Scientific Industries bead beater; Scientific Industries, Bohemia, N.Y.) for three minutes.

Following bead beating, 20 μl of a 20 mg/ml Proteinase K solution (Roche PCR-grade Proteinase K) was added to the bead mixture, mixed by vortexing, and incubated for 10 min at 65° C. Next, 400 μl of Buffer AL (Qiagen) was added to the bead mixture and gently mixed by vortexing. The bead mixture was incubated at 70° C. for 10 minutes. From this resulting mixture, DNA was isolated using a standard Qiagen miniprep kit as described below.

Briefly, 400 μl of 100% ethanol was added to the sample/bead mixture, mixed by vortexing, and the liquid added to a QIAamp Spin Column (in a 2 ml collection tube) and subjected to centrifugation at full speed (20,000×g; 14,000 rpm) for 1 minute. The column was then washed wtih 500 μl of Buffer AW1, followed by 500 μl of Buffer AW2. Extracted DNA trapped in the column was isolated by passing 50 μl of Buffer AE through the column into a clean microcentrifuge tube.

### Example 2

### Preparation of Polynucleotide Array Membranes

The following materials were used in the studies described below: deionized water; sodium carbonate (NaHCO₃; JT Baker Ref: 3506-01; JT Baker, Phillipsburg, N.J.); 1 N sodium hydroxide (NaOH; VWR Ref: VW3222-1); 20×SSPE (G Biosciences Ref: R022; G Biosciences, St. Louis, Mo.); 20% sodium dodecyl sulfate (SDS) (Omni Pur Ref:7990); 0.5 M EDTA (Omni Pur Ref: 4055); Biodyne C (Pall Corp. P/N60251; Pall Corp, East Hills, N.Y.); EDAC (Sigma Ref: E7750-25G); amino-link oligos in water (100 pmol/μl); blue ink 1:100 in water from Cross fountain pen cartridge; and black ink 1:100 in water from Cross fountain pen cartridge.

To prepare polynucleotide blots for use in detection of microorganisms, first, a Biodyne C membrane was incubated in an EDAC solution for 15 minutes with gentle tilting in polypropylene dish. Following the incubation, the membrane was rinsed and washed with water for two minutes. The membrane was then placed in the MN45 miniblotter (following blotter instructions). The residual water was aspirated from the blotter slots using a vacuum or pipette.

Various amino-linked polynucleotides were diluted to a final concentration of 1 pmol/μl in 500 mM NaHCO3 (pH 8.4). Each of the slots of the blotter cassette were filled with 150 μl of the amino-linked polynucleotides and incubated on the membrane for at least 2 minutes.

Each of the polynucleotides were removed and discarded in the order they were applied (thus allowing for approximately the same amount of time in contact with the membrane). The membrane was removed from the blotter and washed in 100 ml of 100 mM NaOH for 8 minutes. The membrane was rinsed quickly using 50 ml of 2×SSPE/0.1% SDS at 60° C. Next, the blot was washed once in 200 ml of 2×SSPE/0.1% SDS for 5 minutes at 60° C. The membrane was also washed in 100 ml of 20 mM EDTA pH 8.0 for 15 minutes at room temperature.

### Example 3

### Hybridization of Nucleic Acid to Polynucleotide Array Blots and Detection Thereof

The following materials were used in the studies described below: blot with polynucleotide stripes (see above); microtiter sealing sheets; 20×SSC (Geno Tech Ref: R019 1L, Geno Tech, St. Louis, Mo.); 20% SDS; NaOH; 0.5M EDTA; 10% sarkosyl solution; DIG wash/block buffer set (Roche Ref:11585762001); BCIP/NBT (Immunetics Ref: CC-S001-030); Anti-digoxigenin alkaline phosphatase antibodies (Roche Ref: 11093274910). The reagents were: 50 ml nucleic acid hybridization buffer (5×SSPE, 0.1% N-laurylsarcosine, 0.02% SDS, 1% block); 50 ml hybridization wash buffer (0.25×SSC, 0.1% SDS) 37° C.; 400 mM NaOH/10 mM EDTA; 1× Roche wash buffer; 1:5000 anti-digoxigenin antibody solution (0.5 ml 10× Maleic acid+0.5 ml 10× Block+4 ml water+1 μl antibody solution); detection buffer (1 ml 10× stock+9 ml water).

The waterbath was preheated to 50° C. and the CodaXcel (Immunetics, Boston) was preheated for two hours in a thermal rocker to 50° C. Ten μl of labeled-PCR products of each amplification (of extracted DNA) were transferred to a separate well of an 8-count PCR strip tube set. Next, the labeled-PCR products were denatured by adding 10 μl of NaOH/EDTA to the 10 μl of PCR product (PCR strip). The membrane was incubated for approximately 5 to 10 minutes in approximately 25 ml of nucleic acid hybridization buffer at 50° C. The denatured PCR products were diluted with 480 μl of nucleic acid hybridization buffer. The CodaXcel (Immunetics) filter was soaked in nucleic acid hybridization buffer, placed in the cartridge with the membrane. The cartridge was closed and sealed using a gasket and screw-tightened lid. Each of the denatured labeled-PCR product mixtures (500 μl) were added to a separate lane of the CodaXcel cartridge and the opening of the cartridge covered (with an adhesive mitrotiter plate seal). The amplicon mixture was hybridized to the membrane for 60 minutes at 50° C. on CodaXcel with shaking in a thermal rocker. The membrane was washed by adding 1 ml of wash buffer (at room temperature) and aspirating the buffer through the membrane. The membrane was further washed five times for 1.5 minutes each using 0.5 ml of hybridization wash buffer at 37° C.

To detect the binding of the amplicons to the polynucleotide-bound membrane, the membrane was first washed for 1 minute with 0.5 ml (per lane) of 1× Roche wash buffer. The membrane was then incubated with 0.5 ml/lane of anti-digoxigenin antibody solution (diluted 1:5,000) for 30 minutes. Following the incubation, the membrane was washed twice with 0.5 ml/lane with 1× Roche buffer for 5 minutes. The membrane was then equilibrated using 1× detection buffer for 1 minute. Next, 0.75 mL/lane of BCIP/NBT reagent was applied to the membrane and incubated for 45 minutes. Following the incubation, the membrane was washed three times with water and then dried.

### Example 4

### Detection of Antibiotic Resistant Bacteria

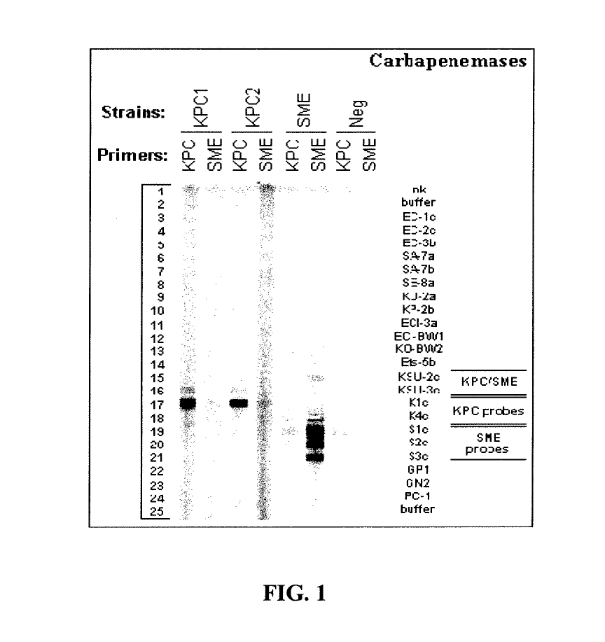

Klebsiella pneumonia containing KPC-1 carbapenemase, K. oxytoca containing KPC-2 carbapenemase, and Serratia marcescens containing SME carbapenemase were obtained from the Centers for Disease Control (CDC). Primer sequence sets: IRS1F AACGGCTTCATTTTTTGTTTAG (SEQ ID NO:40) and IRS2R GCTTCCGCAATAGTTTTATCA (SEQ ID NO:41); or KPC5F TGTCACTGTATCGCCGTC (SEQ ID NO:42) and KPC10R GTCAGTGCTCTACAGAAAACC (SEQ ID NO:43) were used to amplify and label (digoxigenin) extracted DNA (see Example 1). The labeled amplicons were then hybridized to detection oligonucleotides of various lengths and compositions designed to bind with various affinities and to different parts of the amplicon. As shown in FIG. 1, the labeled-amplicons specifically bound to the corresponding detection oligonucleotide. Binding affinities varied, but in each case at least one detection oligonucleotide bound with high affinity (e.g., row 17 for KPC or row 19 for SME; FIG. 1). In no case was there significant hybridization to a non-homologous target. Oligonucleotides with partial homology (rows 15 and 16; FIG. 1) hybridized poorly, indicating high specificity under these conditions.

Two E. coli strains containing ESBL TEM alleles (TEM-10 and TEM-26) were obtained from the American Type Culture Collection (ATCC; Manassass, Va.), and DNA extracted (as above). The extracted DNA was amplified with primers bracketing the entire TEM open reading frame (TEM1_fw ATGAGTATTCAACATTTTCGTGTCGCC (SEQ ID NO:44) and TEM1_rv TTACCAATGCTTAATCAGTGAGGCACC (SEQ ID NO:45), and then detected as described above with the following detection oligonucleotide:: Tem104K_{—}17 ACTTGGTTAAGTACTCA (SEQ ID NO:46), Tem104K_{—}19 GACTTGGTTAAGTACTCAC (SEQ ID NO:47), Tem104K_{—}21 TGACTTGGTTAAGTACTCACC (SEQ ID NO:48), Tem104E_{—}17 ACTTGGTTGAGTACTCA (SEQ ID NO:49), Tem104E_{—}19 GACTTGGTTGAGTACTCAC (SEQ ID NO:50), and Tem104E_{—}21 TGACTTGGTTGAGTACTCACC (SEQ ID NO:51), which were attached to Biodyne™ C membrane as described above. Wild type and mutant detection oligonucleotides of various sizes were designed to bind specifically to each allele. S. aureus with 23S detection oligonucleotide is included as a positive control.

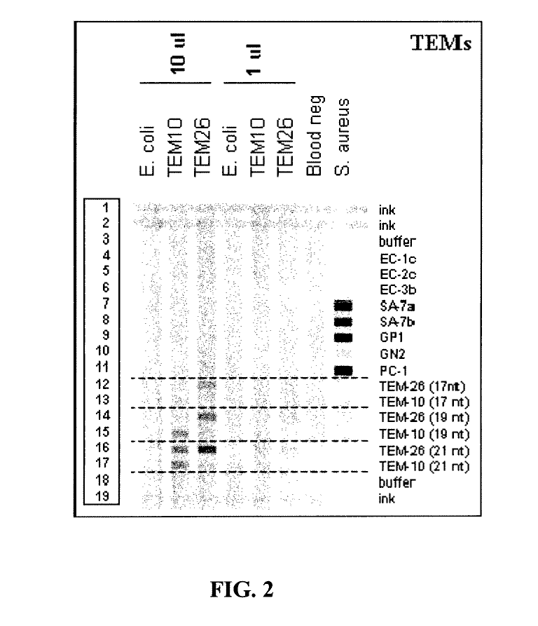

As shown in FIG. 2 (right), the TEM detection oligonucleotide of optimal length for these hybridization conditions was 19 nucleotides, and provided clear identification of the TEM plasmid (vs. E. coli lacking TEM, lane 1; FIG. 2). The oligonucleotide also specifically identified each of the ESBL mutations (rows 14 and 15; FIG. 2). Longer oligonucleotide capable of tolerating mismatches are also designed.

As shown in FIG. 2, the detection of TEM plasmid was stronger when 10 ml of bacterial sample was extracted as compared to 1 ml of sample extracted.

### Example 5

### Detection of C. difficile

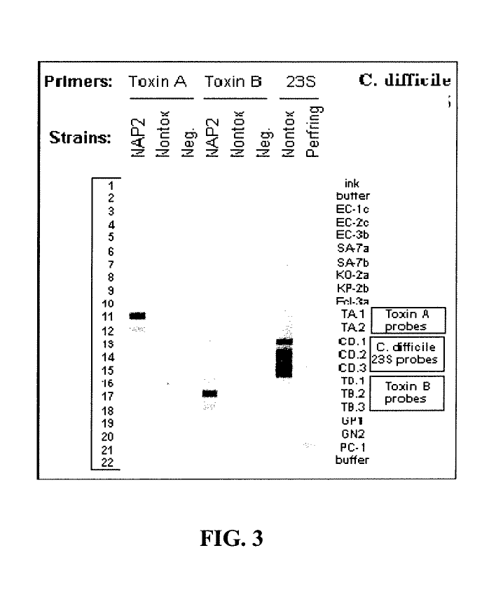

Several C. difficile strains were obtained from the CDC for testing, including the toxigenic “NAP2” strain (toxin A+ and toxin B+) and a nontoxigenic strain (toxin A− and toxin B−). Cultures of the bacteria were prepared and extracted as described above. Primers: Unmodified TB_F1 GAGCTGCTTCAATTGGAGAGA (SEQ ID NO:52), TA_F1 ATGATAAGGCAACTTCAGTGG (SEQ ID NO:53); and 5′ Digoxigenin modification TB_R2 GTAACCTACTTICATAACACCAG (SEQ ID NO:54) and TA_R2 TAAGTTCCTCCTGCTCCATCAA (SEQ ID NO:55) were chosen for amplification of each toxin gene in the extracted DNA. Various detection oligonucleotides were designed (CD.3 GGTATCGTAATTGAAGAGGTTTGG (SEQ ID NO:8), TA.1 GGTGGGAAACTGGAGCAGTTCC (SEQ ID NO:9), and TB.2 TTCAATTCTGATGGAGTTATGCA (SEQ ID NO:10) and used to prepare polynucleotide arrays as described above. The arrays were used to detect the presence of the specific bacterial DNA amplified (as described above). In each case, at least one detection oligonucleotide effectively hybridized to the desired amplicon (FIG. 3, e.g., row 11 for ToxA, row 17 for ToxB), and there was no cross-hybridization to toxin-negative strains. In addition, 23S detection oligonucleotides for identification of C. difficile vs. other Clostridia were designed. These specifically hybridized to C. difficile, clearly differentiating it from C. perfringens (FIG. 3, lanes 7 vs. 8).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit, and scope of the present invention. All such modifications are intended to be within the scope of the present invention.

### Example 6

### An Internal Control

An internal control (IC) is useful for molecular diagnostic assays. Adding IC to the assay allows the user to verify correct performance of one or more parts of the assay, including cell isolation, cell lysis, DNA collection, DNA purification (removal of inhibitors), amplification (e.g., PCR), labeling, hybridization, and detection (e.g., antibody binding and color reaction). Internal controls frequently consist of nucleic acid polynucleotides such as purified plasmid DNA or RNA transcripts. These suffer from the disadvantage of not providing information for the cell isolation and cell lysis steps, and may also be susceptible to instability due to nuclease attack. For viral RNAs these disadvantages have been addressed by encapsulating the RNA in a viral coat. Another disadvantage of traditional purified nucleic acid controls is that they frequently contain bacterial DNA, for example from the E. coli from which the plasmid DNA was isolated, or from the enzymes (e.g., Taq DNA polymerase, T7 RNA polymerase) used to manufacture the RNA transcript. This contaminating DNA may casue false positives in assays designed to detect bacterial DNA.

We have constructed a control for use in the present methods. It contains the following features: (1) primer binding sites derived from 23S ribosomal RNA (recognized by SEQ ID NO:29 and SEQ ID NO:30); (2) heterologous DNA, flanked by the 23S primer sites, consisting of a portion of the NodA gene from S. meliloti; (3) the construct containing NodA flanked by 23S binding sites is inserted into the polylinker site of the yeast integrating vector pRS306 (Sikorski and Hieter, Genetics 122:19-27, 1989); and (4) the integrating vector containing the construct is inserted into the chromosome of the yeast S. cerevisiae (baking yeast) by directing recombination at the URA3 locus using methods known by those skilled in the art (Sikorski and Hieter, supra).

The NodA gene is a “molecular signature” for rhizobial bacteria, which establish symbiotic partnerships with plant roots, and are not expected to ever be found in a human or animal pathogen (Chen et al., J. Bacteriol. 185:7266-7272, 2003). This construct was made by amplifying the S. meliloti genomic gene with the following primers:

23SFE + Nfwd
(SEQ ID NO: 56)
GCGATTTCCGAATGGGGAAACCCATGTACCTGGCGGCCATTCGTTCAAC
23SR + Nrev
(SEQ ID NO: 57)
TTCGCCTTTCCCTCACGGTACTGGAAAATCAGCTGGAACGTGCAGACC

If the resulting yeast cell is added to the assay (e.g., added to the patient blood sample), it will be detected by NodA detection oligos at the end of the assay if all steps of the assay have been successful. When internal control (IC) yeast is added to blood and the assay is performed as described in the preceding sections, the DNA is detected by the NodA detection oligos (even at concentrations as low as 70 cfu/ml). In the absence of the internal control (wild type S. cerevisiae), no NodA is detected.

Because the control is in a yeast cell, there is no added bacterial DNA except for the primer binding sites and sequences used to construct the pRS306 plasmid vector. The absence of E. coli ribosomal DNA is evidenced by the lack of detection by the E. coli (EC) detection oligos, which were present on the same blot as the NodA detection oligos.

1. A method for identifying a microorganism, if present, in one or more samples, in parallel, the method comprising:
providing a first nucleic acid sample from a first source;
providing a second nucleic acid sample from a second source;
amplifying, if present, at least one selected region of nucleic acid sequence in each of the first and second nucleic acid samples, thereby generating amplified first and second nucleic acids;
providing an array of detection oligonucleotides, wherein at least one oligonucleotide hybridizes to an amplified first or second nucleic acid when a sequence that is complementary to the oligonucleotide is present in the amplified first or second nucleic acid;
contacting the array with the amplified first and second nucleic acids; and
performing an assay to detect hybridization between one or more of the detection oligonucleotides on the array and one or more of the amplified first and second nucleic acids, wherein hybridization generates a hybridization pattern with respect to the detection oligonucleotides and thereby identifies a microorganism in the first source or the second source.

2.-44. (canceled)

45. An isolated polynucleotide sequence consisting of any one of SEQ ID NOS: 32 or 34-39 or a sequence complementary thereto or a functionally active variant at least 80% identical to any one of SEQ ID NOs:1-32 or 34-39 or a sequence complementary thereto.

46. The isolated polynucleotide of claim 45, further comprising a heterologous nucleotide sequence.

47. A composition comprising a plurality of polynucleotides immobilized on a solid support, wherein the plurality comprises at least two of the polynucleotides of claim 45.

48. The composition of claim 47, wherein the solid support is a membrane.

49. A kit for use in detecting and/or identifying a nucleic acid of a microorganism and thereby detecting and/or identifying the microorganism, the kit comprising the composition of claim 47 and instructions for use.

50. The kit of claim 49, further comprising a broad-range primer set.

51. The kit of claim 50, wherein the broad-range primer set binds to a region of DNA present in more than one bacterial microorganism or more than one fungal microorganism.

52. The kit of claim 51, wherein the broad-range primer set comprises SEQ ID NO:40 or 41 or SEQ ID NOs:34-39.

53. An isolated fungal cell comprising a vector comprising an exogenous nucleic acid sequence flanked at both the 5′ and 3′ ends by a nucleic acid sequence encoding all or part of a bacterial 23S rRNA.

54. The isolated fungal cell of claim 53, wherein the exogenous nucleic acid sequence is integrated into the genome of the fungal cell.

55. The isolated fungal cell of claim 53, wherein the exogenous nucleic acid sequence comprises all or part of a bacterial NodA gene.

56.-60. (canceled)

61. An isolated fungal cell comprising a vector comprising all of part of a bacterial NodA gene.

62. The isolated fungal cell of claim 61, wherein the NodA gene is flanked both at 5′ and 3′ end by a nucleic acid sequence encoding all or part of a bacterial 23S rRNA.

63. A kit comprising the isolated fungal cell of claim 53 and instructions for extracting nucleic acid from the cell.

64.-65. (canceled)

66. A method for identifying a microorganism, if present, in one or more samples, in parallel, and selecting a treatment regimen, the method comprising:
providing a first nucleic acid sample from a first source;
providing a second nucleic acid sample from a second source;
amplifying, if present, at least two selected regions of nucleic acid sequence in each of the first and second nucleic acid samples, wherein one selected region is a nucleic acid sequence selectively found in a given microbe and one selected region is a nucleic acid sequence conferring antibiotic resistance on the given microbe, thereby generating amplified first and second nucleic acids;
providing an array of detection oligonucleotides, wherein at least one oligonucleotide hybridizes to an amplified first or second nucleic acid when a sequence that is complementary to the oligonucleotide is present in the amplified first or second nucleic acid;
contacting the array with the amplified first and second nucleic acids; and
performing an assay to detect hybridization between one or more of the detection oligonucleotides on the array and one or more of the amplified first and second nucleic acids, wherein hybridization generates a hybridization pattern with respect to the detection oligonucleotides and thereby identifies a microorganism in the first source or the second source and identifies antibiotic(s) to which the microorganism is resistant.


SEQUENCE LISTING (57 sequences)

SEQ ID NO: 1 - DNA, 24 bp, Artificial Sequence
  Description of Artificial Sequence Synthetic oligonucleotide
gaatacataggttaacgaggcgaa

SEQ ID NO: 2 - DNA, 18 bp, Artificial Sequence
  Description of Artificial Sequence Synthetic oligonucleotide
gcgtctggaaagtcgcag

SEQ ID NO: 3 - DNA, 25 bp, Artificial Sequence
  Description of Artificial Sequence Synthetic oligonucleotide
tacatagcatatcagaaggcacacc

SEQ ID NO: 4 - DNA, 27 bp, Artificial Sequence
  Description of Artificial Sequence Synthetic oligonucleotide
ggactgcgatataggattaatcattat

SEQ ID NO: 5 - DNA, 27 bp, Artificial Sequence
  Description of Artificial Sequence Synthetic oligonucleotide
cccattaagttatgtgtgttttagtgg

SEQ ID NO: 6 - DNA, 25 bp, Artificial Sequence
  Description of Artificial Sequence Synthetic oligonucleotide
agyttrctyntyggggttgtaggac

SEQ ID NO: 7 - DNA, 23 bp, Artificial Sequence
  Description of Artificial Sequence Synthetic oligonucleotide
ggaaaagaaatcaaccgagattc

SEQ ID NO: 8 - DNA, 24 bp, Artificial Sequence
  Description of Artificial Sequence Synthetic oligonucleotide
ggtatcgtaattgaagaggtttgg

SEQ ID NO: 9 - DNA, 22 bp, Artificial Sequence
  Description of Artificial Sequence Synthetic oligonucleotide
ggtgggaaactggagcagttcc

SEQ ID NO: 10 - DNA, 23 bp, Artificial Sequence
  Description of Artificial Sequence Synthetic oligonucleotide
ttcaattctgatggagttatgca

SEQ ID NO: 11 - DNA, 19 bp, Artificial Sequence
  Description of Artificial Sequence Synthetic oligonucleotide
ttgcatgctgctctctcgg

SEQ ID NO: 12 - DNA, 25 bp, Artificial Sequence
  Description of Artificial Sequence Synthetic oligonucleotide
taggataagtgcaaagaaatgtggc

SEQ ID NO: 13 - DNA, 21 bp, Artificial Sequence
  Description of Artificial Sequence Synthetic oligonucleotide
gaattgcgttggaatgtggca

SEQ ID NO: 14 - DNA, 20 bp, Artificial Sequence
  Description of Artificial Sequence Synthetic oligonucleotide
tgcaggagaaggggttctgg

SEQ ID NO: 15 - DNA, 25 bp, Artificial Sequence
  Description of Artificial Sequence Synthetic oligonucleotide
cgatacttgttatctaggatgctgg

SEQ ID NO: 16 - DNA, 21 bp, Artificial Sequence
  Description of Artificial Sequence Synthetic oligonucleotide
caccgtcatgcctgttgtcag

SEQ ID NO: 17 - DNA, 24 bp, Artificial Sequence
  Description of Artificial Sequence Synthetic oligonucleotide
acctaatgtcatacctgagccttt

SEQ ID NO: 18 - DNA, 24 bp, Artificial Sequence
  Description of Artificial Sequence Synthetic oligonucleotide
gcagcagaagccatatcacctaat

SEQ ID NO: 19 - DNA, 19 bp, Artificial Sequence
  Description of Artificial Sequence Synthetic oligonucleotide
actgcgttgtgggacgaca

SEQ ID NO: 20 - DNA, 22 bp, Artificial Sequence
  Description of Artificial Sequence Synthetic oligonucleotide
actgcgacgtgggactttaaaa

SEQ ID NO: 21 - DNA, 22 bp, Artificial Sequence
  Description of Artificial Sequence Synthetic oligonucleotide
aaagcagccaagggaatagaag

SEQ ID NO: 22 - DNA, 23 bp, Artificial Sequence
  Description of Artificial Sequence Synthetic oligonucleotide
aggtactacctgttacccgcatc

SEQ ID NO: 23 - DNA, 25 bp, Artificial Sequence
  Description of Artificial Sequence Synthetic oligonucleotide
gtaatacctgttacccacatctgtt

SEQ ID NO: 24 - DNA, 22 bp, Artificial Sequence
  Description of Artificial Sequence Synthetic oligonucleotide
cgaaacggcaggagggcaaacc

SEQ ID NO: 25 - DNA, 22 bp, Artificial Sequence
  Description of Artificial Sequence Synthetic oligonucleotide
atgagaaggaagacgcagtgaa

SEQ ID NO: 26 - DNA, 24 bp, Artificial Sequence
  Description of Artificial Sequence Synthetic oligonucleotide
acgtgggactttaaaaggatagaa

SEQ ID NO: 27 - DNA, 24 bp, Artificial Sequence
  Description of Artificial Sequence Synthetic oligonucleotide
aagagcctcgtatttgaaattcac

SEQ ID NO: 28 - DNA, 19 bp, Artificial Sequence
  Description of Artificial Sequence Synthetic oligonucleotide
gcgattgccttagtagcgg

SEQ ID NO: 29 - DNA, 18 bp, Artificial Sequence
  Description of Artificial Sequence Synthetic oligonucleotide
agcgaaacggcaggaggg

SEQ ID NO: 30 - DNA, 22 bp, Artificial Sequence
  Description of Artificial Sequence Synthetic oligonucleotide
cttaatgaaacggcgcaacacg

SEQ ID NO: 31 - DNA, 22 bp, Artificial Sequence
  Description of Artificial Sequence Synthetic oligonucleotide
ccaactgcggccaccctcaaat

SEQ ID NO: 32 - DNA, 22 bp, Artificial Sequence
  Description of Artificial Sequence Synthetic oligonucleotide
gcgtggaagggagatcggcgtt

SEQ ID NO: 33 - DNA, 20 bp, Artificial Sequence
  Description of Artificial Sequence Synthetic oligonucleotide
tttttttttttttttttttt

SEQ ID NO: 34 - DNA, 18 bp, Artificial Sequence
  Description of Artificial Sequence Synthetic primer
gactccttggtccgtgtt

SEQ ID NO: 35 - DNA, 34 bp, Artificial Sequence
  Description of Artificial Sequence Synthetic primer
gagtgaaaaagtacgtgaaattgttgaaagggaa

SEQ ID NO: 36 - DNA, 34 bp, Artificial Sequence
  Description of Artificial Sequence Synthetic primer
cccgctgaacttaagcatatcaataagcggagga

SEQ ID NO: 37 - DNA, 26 bp, Artificial Sequence
  Description of Artificial Sequence Synthetic primer
gactccttggtccgtgtttcaagacg

SEQ ID NO: 38 - DNA, 24 bp, Artificial Sequence
  Description of Artificial Sequence Synthetic primer
gcgatttcygaaygggggraaccc

SEQ ID NO: 39 - DNA, 21 bp, Artificial Sequence
  Description of Artificial Sequence Synthetic primer
ttcgcctttccctcacggtat

SEQ ID NO: 40 - DNA, 21 bp, Artificial Sequence
  Description of Artificial Sequence Synthetic primer
aacggcttcatttttgtttag

SEQ ID NO: 41 - DNA, 21 bp, Artificial Sequence
  Description of Artificial Sequence Synthetic primer
gcttccgcaatagttttatca

SEQ ID NO: 42 - DNA, 18 bp, Artificial Sequence
  Description of Artificial Sequence Synthetic primer
tgtcactgtatcgccgtc

SEQ ID NO: 43 - DNA, 21 bp, Artificial Sequence
  Description of Artificial Sequence Synthetic primer
gtcagtgctctacagaaaacc

SEQ ID NO: 44 - DNA, 27 bp, Artificial Sequence
  Description of Artificial Sequence Synthetic primer
atgagtattcaacattttcgtgtcgcc

SEQ ID NO: 45 - DNA, 27 bp, Artificial Sequence
  Description of Artificial Sequence Synthetic primer
ttaccaatgcttaatcagtgaggcacc

SEQ ID NO: 46 - DNA, 17 bp, Artificial Sequence
  Description of Artificial Sequence Synthetic oligonucleotide
acttggttaagtactca

SEQ ID NO: 47 - DNA, 19 bp, Artificial Sequence
  Description of Artificial Sequence Synthetic oligonucleotide
gacttggttaagtactcac

SEQ ID NO: 48 - DNA, 21 bp, Artificial Sequence
  Description of Artificial Sequence Synthetic oligonucleotide
tgacttggttaagtactcacc

SEQ ID NO: 49 - DNA, 17 bp, Artificial Sequence
  Description of Artificial Sequence Synthetic oligonucleotide
acttggttgagtactca

SEQ ID NO: 50 - DNA, 19 bp, Artificial Sequence
  Description of Artificial Sequence Synthetic oligonucleotide
gacttggttgagtactcac

SEQ ID NO: 51 - DNA, 21 bp, Artificial Sequence
  Description of Artificial Sequence Synthetic oligonucleotide
tgacttggttgagtactcacc

SEQ ID NO: 52 - DNA, 21 bp, Artificial Sequence
  Description of Artificial Sequence Synthetic primer
gagctgcttcaattggagaga

SEQ ID NO: 53 - DNA, 21 bp, Artificial Sequence
  Description of Artificial Sequence Synthetic primer
atgataaggcaacttcagtgg

SEQ ID NO: 54 - DNA, 23 bp, Artificial Sequence
  Description of Artificial Sequence Synthetic primer
gtaacctactttcataacaccag

SEQ ID NO: 55 - DNA, 22 bp, Artificial Sequence
  Description of Artificial Sequence Synthetic primer
taagttcctcctgctccatcaa

SEQ ID NO: 56 - DNA, 49 bp, Artificial Sequence
  Description of Artificial Sequence Synthetic primer
gcgatttccgaatggggaaacccatgtacctggcggccattcgttcaac

SEQ ID NO: 57 - DNA, 48 bp, Artificial Sequence
  Description of Artificial Sequence Synthetic primer
ttcgcctttccctcacggtactggaaaatcagctggaacgtgcagacc